(19) United States
(12) Patent Application Publication
(10) Pub. No.: US 2013/0325121 A1
(43) Pub. Date: Dec. 5, 2013

(54) PROTEIN BASED MATERIALS, PLASTIC ALBUMIN DEVICES AND RELATED METHODS

(71) Applicant: Clemson University, Clemson, SC (US)

(72) Inventors: Benjamin R. Whatley, Roanoke, VA (US); Xuejun Wen, Henrico, VA (US); Igor Luzinov, Clemson, SC (US); Suraj Sharma, Clemson, SC (US)

(73) Assignee: Clemson University, Clemson, SC (US)

(21) Appl. No.: 13/905,726

(22) Filed: May 30, 2013

Related U.S. Application Data
(60) Provisional application No. 61/653,693, filed on May 31, 2012.
(60) Provisional application No. 61/669,351, filed on Jul. 9, 2012.

(51) Int. Cl. A61F 2/18

(52) U.S. Cl. CPC A61F 2/18
USPC 623/10

(57) ABSTRACT

Medical devices with a plastic albumin body with a defined three dimensional shape that are particularly suitable for pressure equalization tubes. The plastic albumin body can be a unitary substantially monolithic body of albumin. The plastic albumin body has an antibacterial property such that it is resistant to bacterial adhesions and/or bacterial biofilm formation thereon. Methods and processes are described for forming a consolidated protein-based coating on an implant, and coated implants produced therefrom, with improved biocompatibility, nonthrombogenity and antimicrobiality that are suitable for various medical implants and drug delivery devices, such as bone implants, catheters, cannuale, guide wires, stents, shunts, vascular grafts, heart valves, heart and ventricular assist devices, oxygenators, dialyzers, medical devices, and other substrates, such as furniture, keyboards, etc.

### STATEMENT OF FEDERAL SUPPORT

This invention was made with government support under the Department of Defense Grant/Contract Number DR 080572. The United States government has certain rights in this invention.

### RELATED APPLICATIONS

This application claims the benefit of and priority to U.S. Provisional Application Ser. No. 61/653,693, filed May 31, 2012, and U.S. Provisional Application Ser. No. 61/669,351, filed Jul. 9, 2012, the contents of which are hereby incorporated by reference as if recited in full herein.

### FIELD OF THE INVENTION

The invention relates to materials with anti-bacterial properties, and may be particularly suitable for medical uses.

### BACKGROUND

One of the most common health problems among children today is otitis media, or infection within the middle ear. Otitis media is caused by the disruption of normal eustachian tube function, potentially causing permanent ear damage or hearing loss. These issues may also initiate or influence communication problems including slow speech development. Otitis media is usually treated with antibiotics but oftentimes myringotomy is needed. During myringotomy, tympanostomy or a pressure equalization (PE) tube is placed in a respective ear drum to aid in drainage. Unfortunately, an unresolved problem of conventional PE tubes is the accumulation of bacteria at the biomaterial surface and the formation of biofilm. After an implant surface is contaminated with biofilm, it is difficult to use antibiotics due to an increased antibacterial resistance. See, Darouiche et al., J. Infect. Dis. 1994; 3; 720-3. Topical antibiotics (in the form of drops) are used which can help reduce the percentage of viable bacteria. However, there remains a need for alternate biocompatible and/or bacterial resistant materials, particularly materials that are suitable for PE tubes.

Biomaterial-Centered Infection (BCI) is a key factor limiting the optimal utilization of many materials in implants. Despite the use of antibiotics and sterile operating procedures, approximately 112,000 of the 2.6 million orthopedic devices implanted annually in the United States become infected. Bacterial biofilm formation on implant surfaces is frequently the reason for implant failure at least in part because biofilm can be extremely resistant to antibiotic treatments.

Surface characterizations that can decrease bacterial colonization and biofilm formation on implants include positively charged surfaces that may promote bacteria adhesion but prevent biofilm formation; low-surface energy surfaces that provide fluctuating shear forces on the implant; deposition of a protein layer or polymer brushes (arrays of densely end-grafted polymer chains); and antibacterial agent-releasing coatings.

Unfortunately, protein-based coating methods utilized to date form extremely thin coating layers that are often only a monolayer in thickness and the success of such coatings has been limited. Although a protein-based monolayer adsorbed onto an implant surface may decrease bacterial adhesion for a short period of time, the extremely thin coating is not sufficiently stable or robust to prevent biofilm formation. Moreover, when subjected to bodily fluids, protein molecules of a thin adsorbed coating can be destroyed by proteolytic enzymes present in body fluids.

There remains a need for coatings and methods for forming the coatings that are suitable for a wide variety of medical implants and drug delivery devices that exhibit biocompatibility, nonthrombogenity and antimicrobial activity.

### SUMMARY

Embodiments of the invention are directed to implantable medical devices comprising plastic albumin.

Embodiments of the invention are directed to pressure equalization tubes comprising plastic albumin for the treatment of otitis media or other ear-related disorders, injuries or conditions.

Some embodiments are directed to medical devices that include a plastic albumin body having a defined three dimensional shape.

The plastic albumin body can be a unitary substantially monolithic body.

The plastic albumin body can have an antibacterial property such that it is resistant to bacterial adhesions and/or bacterial biofilm formation thereon. The plastic albumin body can include at least one therapeutic agent.

The plastic albumin body can be sized and configured as a biodegradable pressure equalization tube for an ear.

The plastic albumin body can be substantially rigid and can have a substantially medial extending through-channel. The plastic albumin body can have cooperating discrete components.

The plastic albumin can reside on a pre-molded PE tube substrate formed of a different material. The PE tube substrate can comprise TEFLON.

The plastic albumin body can be an overmold on a substrate.

Some embodiments are directed to methods of fabricating a medical device. The methods include: (a) introducing a liquid solution of albumin into a mold having a mold cavity with a defined shape; (b) heating the liquid solution in the mold for a defined time and temperature; and (c) forming a medical device having a solid plastic albumin shape in response to the heating step.

Other embodiments are directed to coating substrates with plastic albumin to provide anti-bacterial protection, e.g., coating surgical devices, surfaces in surgical rooms and/or implants such as PE tubes formed of other materials with plastic albumin.

The introducing step can be carried out using a liquid solution having a concentration of albumin between about 5-100%, typically between about 10-95%, and more typically between about 20-95%. The solution may have a viscosity that is above about 0.005 Pa*s (measured at room temperature).

The heating step can be carried out by placing the mold in an autoclave at between about 80 degrees Celsius and 200 degrees Celsius for between about 10 minutes to several hours, e.g., typically 1-4 hours.

The introducing step can be carried out by providing powder albumin that is dissolved in a liquid.

The method can include applying at least one therapeutic agent onto the albumin, before, during or after the forming step.

Albumin plastic can be used for PE tubes, e.g., albumin plastic can be formed on PE tubes made or other polymers/plastics or the PE tubes can be molded to define the primary substrate material of the PE tubes.

In some embodiments, the PE tubes can be molded and the method can include placing a rod in the mold before the introducing step to be in communication with a respective mold cavity. The introducing step can introduce the liquid under pressure into the mold about the rod, and the forming step can be carried out to forms a through channel in the solid albumin body with a diameter corresponding to a diameter of the rod.

The method can include placing a substrate in the mold before the introducing step to be in communication with a respective mold cavity. The introducing step can introduce the liquid under pressure into the mold about the substrate and the forming step can be carried out to overmold the plastic albumin onto a surface or surfaces of the substrate.

Still other embodiments are directed to method of treating a patient. The methods include: (a) providing a tube with an open channel, the tube comprising a substrate with a plastic albumin coating or overmold or a tube having a plastic albumin substrate defining the tube; (b) placing the tube in a target location of a patient; and (c) providing at least one of a (i) drainage channel (ii) access path or (ii) vent or air path; or (iii) tissue support using the tube.

The placing step can be carried out by placing the tube as a pressure equalization tube in an ear of the patient.

Other methods are directed to treating substrates to provide anti-bacterial properties. The methods include: (a) providing a target substrate; (b) applying liquid albumin to the target substrate; and (c) heating the substrate with the applied liquid albumin to transform the albumin into plastic albumin on the target substrate.

The target substrate can be a pressure equalization (PE) tube.

The target substrate is a device used in a patient or baby care facility.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE FIGURES

FIG. 1 is a top view of an exemplary medical device according to embodiments of the present invention.

FIG. 2 is a side perspective view of an exemplary multi-component body device according to embodiments of the present invention.

FIG. 3 is a side perspective view of another exemplary medical device according to embodiments of the present invention.

FIG. 4A is a side perspective view of yet another exemplary medical device according to embodiments of the present invention.

FIG. 4B is a side view of a catheter according to embodiments of the present invention.

FIG. 5A is a top view of a PE tube according to embodiments of the present invention.

FIG. 5B is a side view of the device shown in FIG. 5A.

FIG. 5C illustrates the PE tube shown in FIGS. 5A and 5B in position according to embodiments of the present invention.

FIG. 6A is a top perspective view of a closed mold according to embodiments of the present invention.

FIG. 6B is a side perspective view of a rod that can be used with a mold to form a medical device with an open through channel according to embodiments of the present invention.

FIG. 7 is an example of a container with a liquid albumin solution according to embodiments of the present invention.

FIG. 8 is a top side view of a portion of a mold configured to mold a plurality of medical devices concurrently according to embodiments of the present invention.

FIG. 9A is a schematic illustration of a substrate that can be introduced into a mold for fabricating a plastic albumin overmold thereon according to embodiments of the present invention.

FIG. 9B is an example of a medical device with a plastic albumin overmold according to embodiments of the present invention.

FIG. 9C is an example of a conventional PE tube of a plastic substrate (e.g., TEFLON) coated with plastic albumin according to embodiments of the present invention.

FIG. 10A is a flow chart of exemplary operations that can be used to fabricate medical devices according to embodiments of the present invention.

FIG. 10B is a flow chart of exemplary operations that can be used to coat substrates with plastic albumin according to embodiments of the present invention.

FIG. 11A is a digital photograph of a conventional PE tube (TEFLON).

FIG. 11B is a digital photograph of a plastic albumin PE tube according to embodiments of the present invention.

FIGS. 12A-12C are digital photographs of live/dead stain showing greater concentration of bacteria on a control (FIG. 12A) and the commercially available PE tube (FIG. 12B) when compared to the albumin plastic (FIG. 12C).

FIG. 13 illustrates a process for forming a protein-based coating material as described herein according to embodiments of the present invention.

FIG. 14 illustrates a process for coating an implantable structure with a protein-based coating material as described herein according to embodiments of the present invention.

FIG. 15 illustrates another process for coating an implantable structure with a protein-based coating material as described herein according to embodiments of the present invention.

FIG. 16 graphically illustrates the differential scanning thermographs (FIG. 16A) and the thermogravimetric analysis (FIG. 16B) of a protein-based coating material in powder form and following compression molding according to embodiments of the present invention.

FIG. 17 graphically illustrates physical characteristics of a protein-based coating material as described herein according to embodiments of the present invention.

FIG. 18 graphically compares the experimental and calculated values for tensile modulus for protein-based coating materials including a variety of add-in levels of a second polymer in a blended composition according to embodiments of the present invention.

FIG. 19 graphically compares the experimental and calculated values for tensile strain for protein-based coating materials including a variety of add-in levels of a second polymer in a blended composition according to embodiments of the present invention.

FIG. 20 graphically compares the experimental and calculated values for tensile stress for protein-based coating materials including a variety of add-in levels of a second polymer in a blended composition according to embodiments of the present invention.

FIG. 21 presents the surface Fourier transform infrared spectroscopy results for a polymer material including a synthetic polymer as the only polymer of the material (FIG. 21A), a polymer material including the synthetic polymer and a protein-based polymer in a blend (FIG. 21B), and a polymer material including the protein-based polymer as the only polymer of the material (FIG. 21C) according to embodiments of the present invention.

FIG. 22 graphically illustrates the contact angle measurements for protein-based coating materials as described herein according to embodiments of the present invention.

FIGS. 23A-23E provide scanning electron micrograph images of protein-based coating materials as described herein according to embodiments of the present invention.

FIG. 24 presents differential scanning thermographs (FIG. 24A) and thermogravimetric analysis (FIG. 24B) of implants as may be coated by the protein-based coating materials as described herein according to embodiments of the present invention.

FIG. 25A and FIG. 25B present views of different magnifications of a titanium substrate pre-treated with a polymeric anchoring layer according to embodiments of the present invention.

FIG. 26A and FIG. 26B present views of different magnifications of a titanium substrate following pretreatment with a polymeric anchoring layer and coated with a protein-based coating layer as described herein according to embodiments of the present invention.

FIG. 27A and FIG. 27B present views of increasing magnification of a titanium substrate following pretreatment with a polymeric anchoring layer and coated with a protein-based coating layer as described herein followed by annealing of the coated substrate according to embodiments of the present invention according to embodiments of the present invention.

FIG. 28 presents a view of a titanium substrate following pretreatment with a polymeric anchoring layer and coated with a protein-based coating layer as described herein followed by annealing of the coated substrate and compression molding of the coated substrate according to embodiments of the present invention according to embodiments of the present invention.

FIG. 29 presents the results of colonization of Staphylococcus aureus onto a protein-based coating material as described herein (FIG. 29A) and an uncoated titanium surface (FIG. 29B) according to embodiments of the present invention.

FIG. 30A illustrates a bone implanted titanium implant coated with a protein-based coating material as described herein according to embodiments of the present invention.

FIG. 30B illustrates a bone implanted with a non-coated titanium implant according to embodiments of the present invention.

FIG. 31 graphically illustrates the release over time of a proteinaceous compound from a protein-based coating material as described herein according to embodiments of the present invention.

FIG. 32 graphically illustrates the release over time of a proteinaceous compound from a protein-based coating material as described herein according to embodiments of the present invention.

FIG. 33 graphically illustrates the release over time of a proteinaceous compound from a protein-based coating material as described herein according to embodiments of the present invention.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The term “medical” use refers to both human, veterinarian and other animal use, both for research and therapeutic applications.

The term “about” means that the specified parameter can vary from its recited number by between +/−20%, and is more typically within +/−10% of that number.

The term “consisting essentially of” refers to a device or a coating that includes at least about 90% plastic albumin by weight of the device or the coating, respectively, with optional supplemental drug coatings and/or constituents.

The term “tissue” refers to soft tissue and hard tissue (e.g., bone).

The term “thin” refers to a thickness that is between about 0.0001 mm (on average) to about 2.0 mm (on average), typically between about 0.1 mm (on average) to about 2.0 mm (on average), and more typically is between about 0.7 mm (on average) to about 1.2 mm (on average).

The term “liquid” includes “gels”.

The terms “plastic” and “plasticized” refer to a solid material (e.g., a solid albumin) that can be formed from a liquid, powder, and/or gel via molding, extrusion and/or other fabrication techniques. A plastic or plasticized albumin can be formed into a desired solid shape, typically into a substantially rigid body or coating on a rigid body, although the plasticized albumin may also be formed into and/or onto semi-rigid and/or flexible solid bodies.

The term “sterile” refers to a device that meets the applicable medical sterility standards for medical applications and use.

The term “albumin” refers to a protein that is water soluble, is moderately soluble in concentrated salt solutions, and experiences heat denaturation. Albumin proteins are commonly found in blood plasma and are unique from other blood proteins in that they are not glycosylated. In certain embodiments, the albumin is a serum albumin, an egg white albumin, and/or a fragment thereof. A fragment can comprise at least 2 contiguous amino acids (e.g., 2, 3, 4, . . . 200 500 . . . 600 or more acid acids). “Albumin” as used herein includes an albumin mutant and/or a fragment thereof. “Mutant” as used herein refers to an albumin which comprises at least one amino acid substitution, insertion, deletion, or any combination thereof (i.e., a mutant can comprise 1, 2, 3, 4, 5, 6, 7, 8, 9, 10, or more amino acid substitutions, insertions, and/or deletions). Substitutions, insertions, and/or deletions can be confined to one location of the protein sequence and/or can be at multiple locations of the protein amino acid sequence.

In some embodiments, gelatin plastic or collagen plastic and a mixture of albumin with other proteins such as gelatin may also be used.

An albumin can be natural (e.g., obtained from the serum of a donor) and/or synthetic (e.g., derived from PCR and/or recombinant DNA techniques) and can be obtained from any suitable source (e.g., mammal, avian, yeast, bacteria, etc.). “Avian” as used herein includes, but is not limited to, chickens, ducks, geese, quail, turkeys, pheasants, ratites (e.g., ostrich), parrots, parakeets, macaws, cockatiels, canaries, and finches. “Mammal” as used herein includes, but is not limited to, primates (e.g., humans), non-human primates (e.g., monkeys, baboons, chimpanzees, gorillas), bovines, ovines, caprines, ungulates, porcines, equines, felines, canines, lagomorphs, pinnipeds, and rodents (e.g., rats, hamsters, and mice). In some embodiments, an albumin can be obtained from a patient's serum and can be for their personal use (e.g., for preparing an implantable medical device to be implanted in the patient). An albumin can be from a donor. In some embodiments, an albumin can be obtained from a human, a bovine, a chicken egg white, or any combination thereof.

“Albumin” as used herein can refer to Cohn Fraction V, but is not limited to the method of obtaining albumin by using the Cohn method of fractionating serum proteins. An albumin can be obtained by any suitable method, including, but not limited to, the Cohn method of fractionating serum proteins, ethanol fractionation methods, heat shock, chromatography, crystallization, charcoal filtration, and any combination thereof. An albumin can also be obtained from commercial sources, such as, but not limited to, Sigma-Aldrich® of St. Louis, Mo.

An albumin can be produced and/or purified by any suitable method known in the art. Such methods include conventional techniques in molecular biology, microbiology, chromatography, and recombinant DNA. These techniques are explained in, for example, Sambrook et al., 2001, Molecular Cloning: A Laboratory Manual, Third Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins, eds.); Transcription and Translation, 1984 (Hames and Higgins, eds.); Animal Cell Culture, 1986 (R. I. Freshney ed.); Immobilized Cells and Enzymes, 1986, (IRL Press); Perbas, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively); Current Protocols in Molecular Biology, John Wiley & Sons, Inc. (1994), Protein Purification: Principles and Practice, Scopes, Robert K., Springer-Verlag (1994), and all more current editions of these publications, each of which are incorporated herein by reference in their entirety.

As recognized by a skilled artisan, nucleic acid sequences and/or amino acid sequences can be obtained through publicly available databases, such as the National Center for Biotechnology Information (NCBI) database or commercially available databases, such as from Celera Genomics, Inc. (Rockville, Md.). Further exemplary albumins include, but are not limited to, NCBI GenBank Accession Nos. AAA98797, CAA00844, CAA76847, CAA43098, and any combination thereof, each of which are incorporated herein by reference in their entirety.

In some embodiments, an albumin solution (i.e., a liquid albumin) can be used for fabricating a medical device or a coating. An albumin solution can have a concentration of albumin from about 1% to about 95% by weight of the solution or any range therein, such as, but not limited to, about 10% to about 90%, about 20% to about 75%, or about 25% to about 50% by weight of the solution. An albumin solution can comprise any suitable solvent, such as, but not limited to, water, saline, a buffer (e.g., a phosphate buffer such as phosphate buffered saline (PBS), a carbonate buffer, a tris(hydroxymethyl)aminomethane (Tris) buffer, etc.), or any combination thereof. In some embodiments, an albumin solution can comprise saline and about 25% to about 90% albumin by weight of the solution.

Optionally, other proteins, such as, but not limited to, gelatin and/or collagen, and/or a processing agent, such as, but not limited to, an excipient (e.g., a sugar and/or an inorganic salt), a surfactant (e.g., an ionic surfactant, a non-ionic surfactant and/or a fluorochemical), and/or a curing agent (e.g., an epoxy, a urethane, and/or a polyester), can be added to an albumin either in dry (e.g., a powder albumin) or liquid (e.g., an albumin solution) form prior to formation of an implantable medical device. In certain embodiments, no additional proteins and/or processing agents are added to an albumin prior to the formation of an implantable medical device. As one of ordinary skill in the art will recognize, the purity of an albumin can vary. Thus a contaminant (e.g., another biomolecule and/or compound) can be present in an albumin, and thus, for example, can be present in an albumin solution. Generally, an albumin has a purity of at least about 70%, 80%, 85%, 90%, 91%, 92%, 93%, 94%, 95%, 96%, 97%, 98%, 98.5%, 99%, 99.5% or more. In certain embodiments, an albumin has a purity of at least about 96% or more.

The term “drug” is used interchangeably with “therapeutic agent” and “biologically active agent”, and refers to an agent (e.g., an organic compound, an inorganic compound, a biomolecule, etc.) that has a beneficial effect on a subject/patient, which beneficial effect can be complete or partial. “Biomolecule” as used herein refers to a protein, a polypeptide, a nucleic acid (e.g., a deoxyribonucleic acid and/or a ribonucleic acid), and/or a fragment thereof. Exemplary drugs include, but are not limited to, analgesics such as non-steroidal anti-inflammatory drugs and opioids; antibiotics; anti-scarring agents; steroids; anti-inflammatory agents such as steroids, salicylates, ibuprofen, naproxen, dexamethasone, prednisolone, corticosterone, budesonide, estrogen, sulfasalazine and mesalamine; bisphosphonates; anti-thrombotic agents such as heparin, heparin derivatives, urokinase, and PPack (dextrophenylalanine proline arginine chloromethylketone); antineoplastic/antiproliferative/anti-miotic agents such as paclitaxel, 5-fluorouracil, cisplatin, vinblastine, vincristine, epothilones, endostatin, angiostatin, angiopeptin, and thymidine kinase inhibitors; anesthetic agents such as lidocaine, bupivacaine and ropivacaine; vascular cell growth promoters such as transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); antimicrobial agents such as triclosan, cephalosporins, aminoglycosides and nitrofurantoin; cytotoxic agents, cytostatic agents and cell proliferation affectors; vasodilating agents; antibodies (e.g., monoclonal antibodies and/or polyclonal antibodies); growth factors; cytokines; hormones; vitamins; minerals; or any combination thereof.

Referring to FIGS. 1-4, the figures, embodiments of the invention are directed to medical devices 10 comprising plastic albumin. The medical devices 10 can be implantable medical devices including internal devices and externally accessible (e.g., subdermal) devices, such as, for example, devices that provide access or drainage ports. The devices 10 are typically three dimensional devices having desired (defined) shapes selected for their end use. The devices 10 may include at least one through channel 11. However, the devices 10 may include a partial length channel or can be devoid of a channel depending on the medical application. The plastic albumin can provide a “natural” biofilm resistance and/or anti bacterial properties that impedes bacterial growth. For example, such natural resistance can be better than conventional PE tubes that incorporate silver particles (see, e.g., FIGS. 12A-12C). However, in some embodiments, the plastic albumin used as coatings and/or overmolds on substrates or to form a primary substrate that defines a shape can also incorporate silver, e.g., particles for additional microbe protection.

The plastic albumin medical devices 10 can be biodegradable over time. The patency of the biodegradable device 10 in the body can vary depending on the thickness and configuration of the device, but can, at least partially, if not substantially, degrade within between about 1 month to 1 year, and more typically between about 2-6 months, such as, for example, 2 months, 3 months, 4 months, 5 months or 6 months. The amount and rate of degradation in vivo can be assessed using accelerated in vitro tests as is known by those of skill in the art.

The devices 10 can optionally include at least one drug incorporated therein and/or thereon providing a therapeutic activity in vivo. In some embodiments, the devices 10 can include, for example, those described above, such as one or more antibiotics, anti-inflammatory agents, anti-scarring agents, and/or steroids.

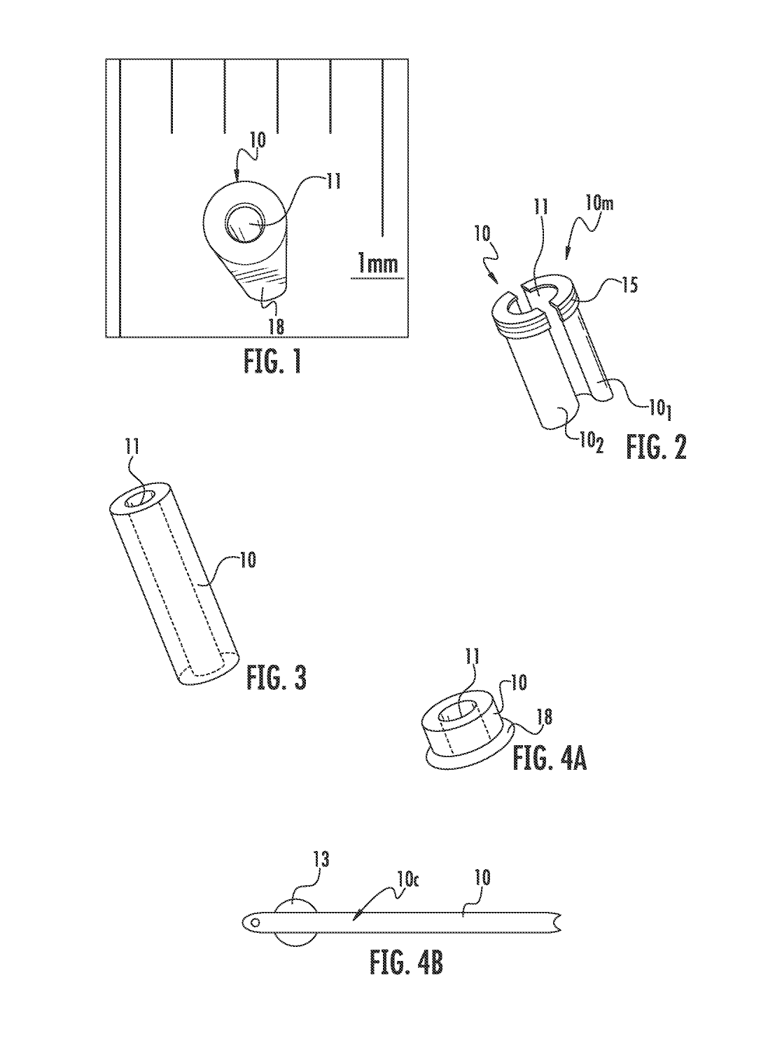

As shown in FIGS. 1, 3 and 4A, 4B the medical devices 10 can comprise a monolithic unitary shaped body of plastic albumin. However, as shown in FIG. 2, the device 10 can be a multi-component substantially monolithic body 10m.

FIG. 2 also illustrates that the multi-component device 10m can optionally include at least one groove 15 that may accommodate a suture (which may also be degradable or restorable). The multi-component body can be press-fit, adhesively attached or otherwise held closely spaced or abutting each other, to hold the components together. The device 10m can include more than one groove (although not shown). The unitary body devices 10 may also optionally include at least one groove which can be used to cooperate with a suture to anchor the device at a target site, but is not required.

FIG. 4A illustrates that the device 10 can include at least one radially outward projecting lip 12 that may be sized to rest against target tissue interface to hold the device in location. If desired, a suture can optionally be positioned proximate the lip and primary body intersection to help anchor the device in situ (not shown). FIG. 2 illustrates that the device 10 can include both a lip 18 and a groove (shown in the lip 18), but the groove may also be placed at other locations.

The multi-component devices 10m may be configured so that their components, shown as component 10₁ and 10₂ in FIG. 2, frictionally and/or matably engage. In other embodiments, components 10₁ and 10₂ may reside (typically closely) spaced apart from each other. In other embodiments, one of the components 10₁ may be used without the other. Indeed, the solid device 10 may be shaped in any form and is not required to be a closed shape.

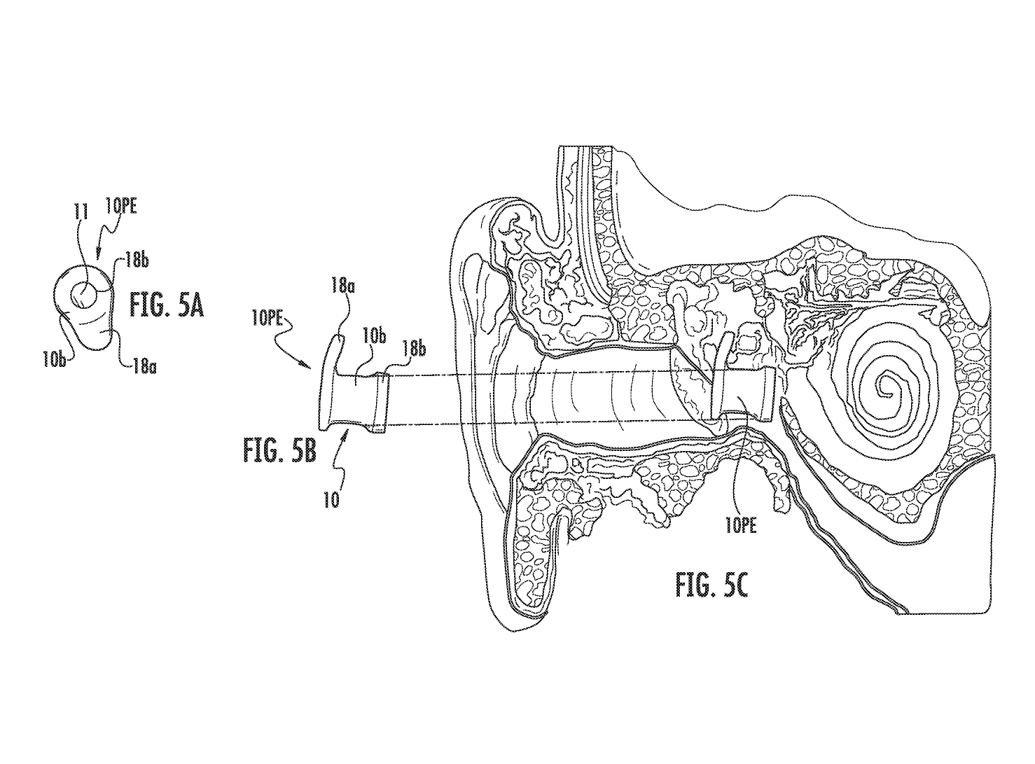

FIGS. 1 and 5A-5C illustrate that the device 10 may be sized and configured as a pressure equalization (PE) tube 10PE for implantation into a patient's ear. When there is an infection or fluid accumulation in the middle ear, a pressure equalization tube can be implanted into the middle ear by inserting the tube through the ear drum to drain the fluid. Current clinical treatments use non-degradable tubes. The tubes are typically required to be removed from the middle ear or they self-eject, the former can disturb the formation of eardrum.

The tubes 10PE can incorporate at least one drug into and/or onto the PE tube thereby serving as a drug delivery vehicle to treat infections and/or promote ear drum formation. In particular embodiments, the tubes 10PE can include at least two different drugs, e.g., both an antibiotic and a steroid such as ciprofloxacin and dexamethasone.

The PE tubes 10PE are preferably biodegradable and can have an efficacious life in vivo of between about 1 month to about 1 year (on average), more typically about 3-6 months.

As shown in FIGS. 5A-5C, the tubes 10PE can include a monolithic unitary plastic albumin body 10b with a first end portion having a large asymmetrical lip 18a and an opposing second end portion that includes a smaller lip (or ledge) 18b. However, other configurations may be used. The tubes 10PE can be designed to be similar to commercial devices. The inner diameter can be about 1 mm, the outer diameter can be between about 2-3 mm, and the length can be about 3-6 mm, more typically between about 4-6 mm. However, other shapes, sizes and dimensions may be used.

Further, although the tube 10PE is shown with one large center channel 11, the tube 10PE can be formed with a plurality of channels having sufficient size to allow drainage.

In other embodiments, the medical devices 10 can be other medical devices such as those used for other medical applications for providing drainage, maintaining openings and/or buttressing or providing support to local structures, including, for post-operative support in nasals after sinus surgeries or rhinoplasty, other plastic surgeries, dental uses (e.g., for post-surgery treatment for excision of wisdom teeth, use with dental implants and the like), for providing support to tendon or ligament repairs, for providing support to bone repairs or fractures, particularly for small bones, such as those in a foot and/or hand, for providing buttressing to sternums post-surgery (such as after open heart surgery to promote healing and provide acute support) for providing “temporary” or “acute” drug access ports or fluid drainage ports, for burn victims for drainage, for trachea openings/access, for prostate access, and the like. In some embodiments, the plasticized albumin can coat or be in the form of a catheter or a portion of a thin-walled catheter 10c, shown as a FOLEY catheter with optional inflatable balloon 13 in FIG. 4B.

In other embodiments, the plastic albumin can be used to coat medical devices 10 or other substrates to provide anti-bacterial properties, including, but not limited to, door knobs, surgical or hospital masks, gloves, gowns, sutures, telephones, toilet seats, keyboards, hospital furniture, including devices and furniture in surgical or hospital or short or long term care rooms, e.g., IV's, hospital beds, rails wheelchairs, bed pans, portable toilets, eyeglasses, face masks, television remote controls, displays, User Interfaces for displays or computers, ventilators, conduits, monitors, displays, sensors, blood pressure cuffs, pumps, needles, saline or medicine bags and the like). The plastic albumin may also be used for other applications including food preparation (kitchens and devices and/or surfaces therein), spas, hot tubs, pedicure bowls or baths, pedicure and manicure tools and the like, baby changing tables in nurseries, daycares, portable diaper bags, and the like.

In some embodiments, methods and processes can form a consolidated protein-based coating on an implant with improved biocompatibility, nonthrombogenity and antimicrobiality that are suitable for various medical implants and drug delivery devices, such as bone implants, catheters, cannuale, guide wires, stents, shunts, vascular grafts, heart valves, heart and ventricular assist devices, oxygenators, dialyzers, and other medical devices, and other substrates, such as furniture, keyboards, etc.

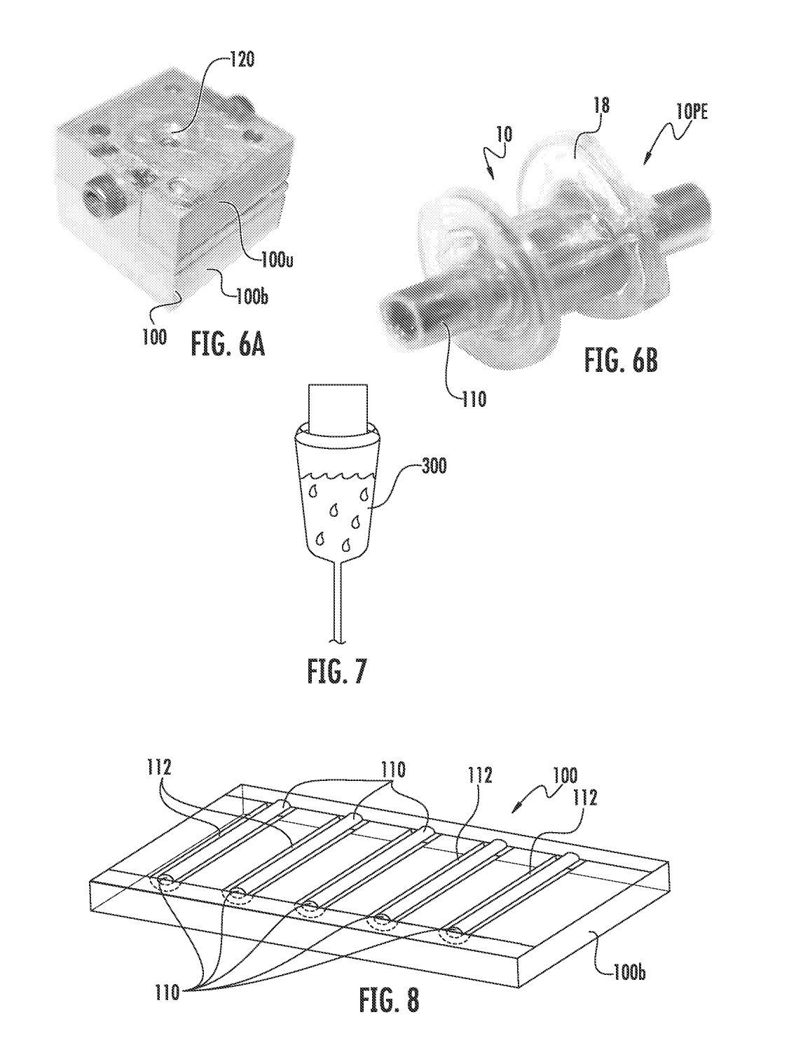

Any suitable mold can be used to form a desired molded plastic albumin device, at least for embodiments where the albumin is used without an underlying substrate and it is desired to form the plastic albumin in a defined shape. However, for completeness, an example of a mold is shown in FIG. 6A. FIG. 6A illustrates a mold 100 that can be used to fabricate a medical device 10, such as a PE tube 10PE. The mold 100 includes matable mold upper and lower shells 100u, 100b with respective internal shaped cavities, each having a concave shape that faces each other to form the outer wall of the tubes 10PE. The mold 100 can be stainless steel and can be in a sterile condition for forming the medical device 10. However, other suitable materials may be used. The device 10 can also be sterilized after it is fabricated by conventional and well-known methods. To form the channel 11, an elongate, small, removable rod 110 (FIG. 6B), which is typically metal, can extend across the mold 100 trapped between the two mold pieces 100u, 100b to create the channel 11 wall with a desired inner diameter for the tubes 10PE. The mold 100 can be configured with two halves with each half being screwed or otherwise releasably attachable together to form the tubes 10PE.

The mold 100 can have at least one port 120 to inject liquid albumin using a syringe, nozzle or other liquid introduction system or device to allow for the pressurized delivery into the mold (e.g., so that the mold can be pressurized). Once the liquid albumin has been injected into the mold 100, the mold 100 can be heated. The mold can be heated for a defined temperature and time. For example, the mold 100 can be placed in an oven or an autoclave at a temperature that is typically between about 80-200 degrees Celsius, and in some embodiments is between about 100-150 degrees Celsius, for a defined time. The time can be between about 15 minutes to several hours, e.g., about 4 hours, typically between about 15 minutes to 1 hour. In some embodiments, the mold 100 can be placed in an autoclave at a temperature of between about 110-130 degrees Celsius for about 15-30 minutes. In other embodiments, other curing techniques may be used, including, for example, combinations of heat (dry or wet), optical (e.g., UV), radiation, and the like.

After curing, the solid (plastic) albumin medical device 10 can have between 5-100% albumin by weight of the device, typically between about 10-99%, including, for example, between about 20-99%, such as about 25%, about 30%, about 35%, about 40%, about 45%, about 50%, about 55%, about 60%, about 65%, about 70%, about 75%, about 80%, about 85%, about 90% or between about 90-99%, albumin by weight of the device (or any number between any of the cited amounts), after molding is complete (after the device is cured).

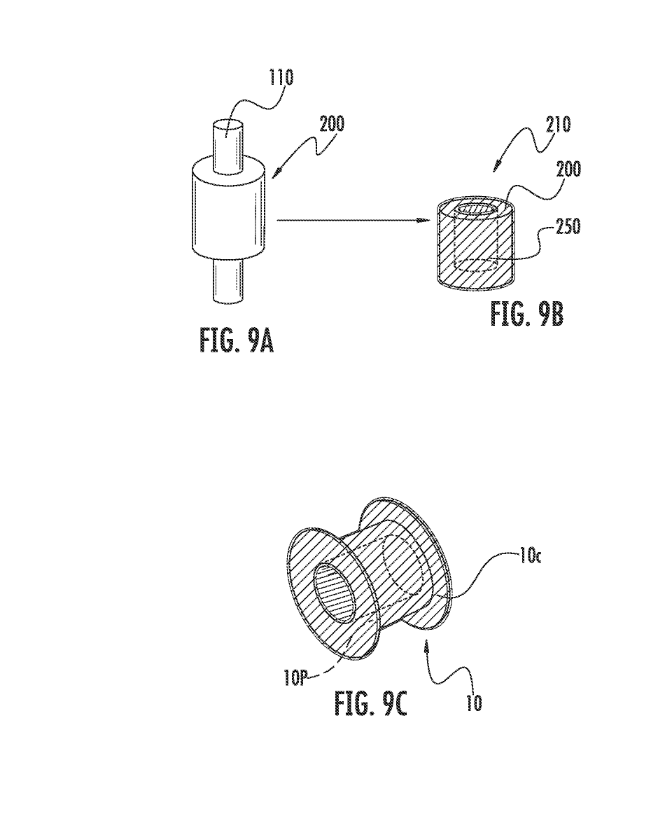

FIG. 8 illustrates an example of a mold bottom 100b with a plurality of concave mold cavities 112 that can mold a plurality of albumin tubes 10PE or other devices 10 concurrently. The top of the mold is not shown, but can have a similar configuration as well as one or more albumin liquid entry ports. FIGS. 9A and 9B illustrate another embodiment. In this embodiment, the medical device 210 includes a plastic albumin overmold layer 250 on a different substrate. The overmold layer 250 is shown on an outer surface of the substrate 200, but it can be on either the inner or outer surfaces (or both) of the other substrate 200. In some embodiments, the overmold layer can encase the underlying substrate. The substrate 200 with optional mold tube 110 can be placed in a mold and the liquid albumin can be introduced to surround or contact the substrate 200 or target areas or volumes thereof. A mold with the mold tube, substrate 200 and the liquid albumin can be heated for a defined time and temperature to form the overmold 250. The plastic albumin overmold 250 can provide antibacterial resistance.

FIG. 9C illustrates that the device 10 can include a primary substrate 10p with a coating of plastic albumin 10c according to some embodiments. As shown, the primary substrate 10p is a pre-formed PE tube of another material to which the plastic albumin is applied.

Referring to FIG. 7, in some embodiments, the liquid albumin 300 can comprise albumin concentrated and/or reconcentrated to between about 5-90% by weight of the solution, typically between about 20-70% by weight of the solution, and more typically between about 25-50% by weight of the solution. The viscosity of the liquid albumin solution 300 formulated for introduction into the mold, at ambient (room) temperature, can be at least about 4 times greater than that of water, and is typically about 5-10× that of water, such as between about 0.005 to about 0.009 Pa*s, and more typically is between about 0.0082 Pa*sec to about 51.81 Pa*s (measured at room temperature). The liquid albumin 300 can comprise any suitable solvent including buffers (e.g., PBS), water and/or saline. Typically, the liquid solution 300 comprises saline. The liquid albumin solution 300 can include other proteins such as collagen, gelatin and the like. The liquid albumin solution can be heated or cooled prior to adding to the mold or may be introduced at room temperature. The liquid albumin solution 300 can be provided as a product having a shelf-life and may be held in a frozen or refrigerated state.

The liquid albumin solution 300 can be formulated from powder albumin and/or liquid albumin solution. If powder, it is dissolved (completely or partially) in a solvent for use. In some embodiments, a liquid albumin solution can be concentrated to a desired concentration and/or viscosity. In some embodiments, a liquid albumin solution can be diluted to a desired concentration and/or viscosity.

As noted above, the plastic albumin medical devices 10 and/or overmold 250 can include a drug. A drug can be loaded and/or integrated in various ways. For example; the fabricated devices 10, 210 (after molding or curing) can be sprayed (coated). The fabricated devices 10, 201 can be immersed into a solution containing one or more drugs to allow the devices 10 to uptake the drugs via diffusion and/or absorption. Alternatively or additionally, because the temperatures that can be used to cure the plastic albumin can be selected to be below the denaturation and/or melting point of a drug, a powder albumin and/or a liquid albumin solution can be mixed with a drug prior to fabrication into a plastic. A drug can be placed, added, and the like into a mold before a liquid albumin is inserted and/or can be pre-mixed with a liquid albumin and introduced together as a single liquid into the mold. In some embodiments, the plastic albumin medical devices 10 comprise an antibiotic, such as, but not limited to, ciprofloxacin. In some embodiments, the plastic albumin medical devices 10 comprise a steroid, such as, but not limited to, dexamethasone.

In some embodiments, the medical devices 10 can be configured to provide a time release of the drugs. It is believed that, where used, the drugs can diffuse out of the plastic albumin before the plastic albumin begins to degrade. However, alternatively or additionally, the drugs may be released as the plastic albumin degrades.

The device 10, 210 can be formed of an albumin material that is biocompatible by cytotoxicity and sensitivity testing specified by ISO (ISO 10993-5 1999: Biological evaluation of medical devices—Part 5: Tests for in vitro cytotoxicity and ISO 10993-10 2002: Biological Evaluation of medical devices-Part 10: Tests for irritation and delayed-type hypersensitivity.

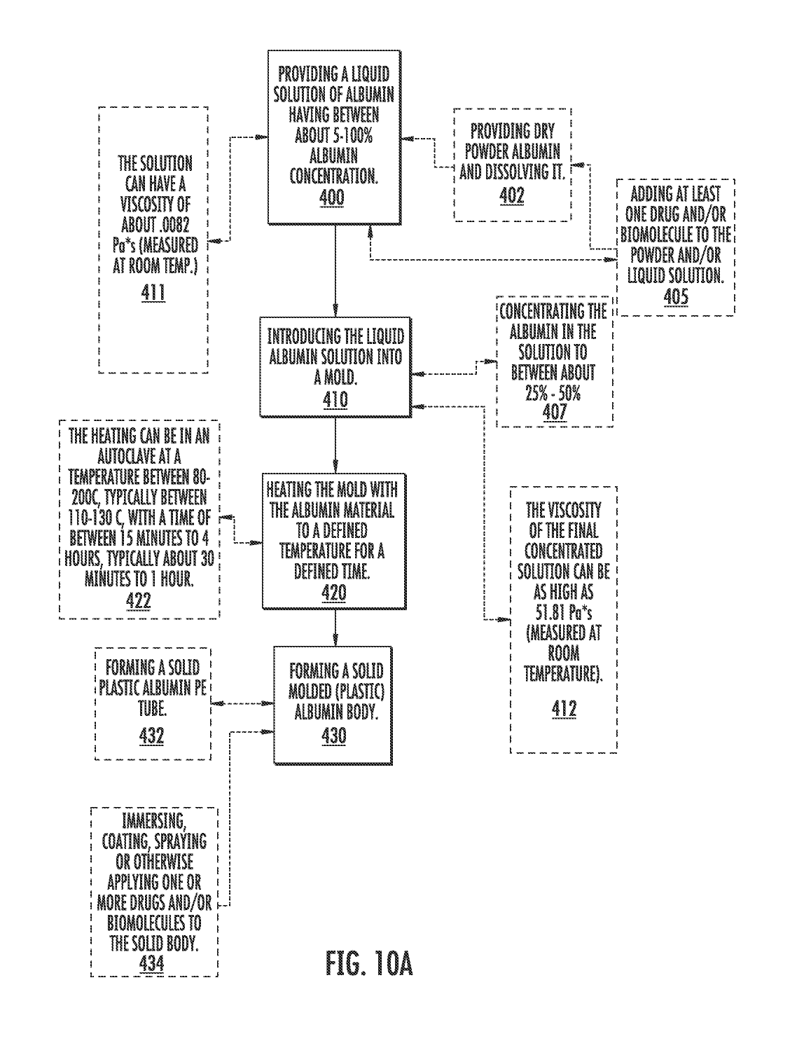

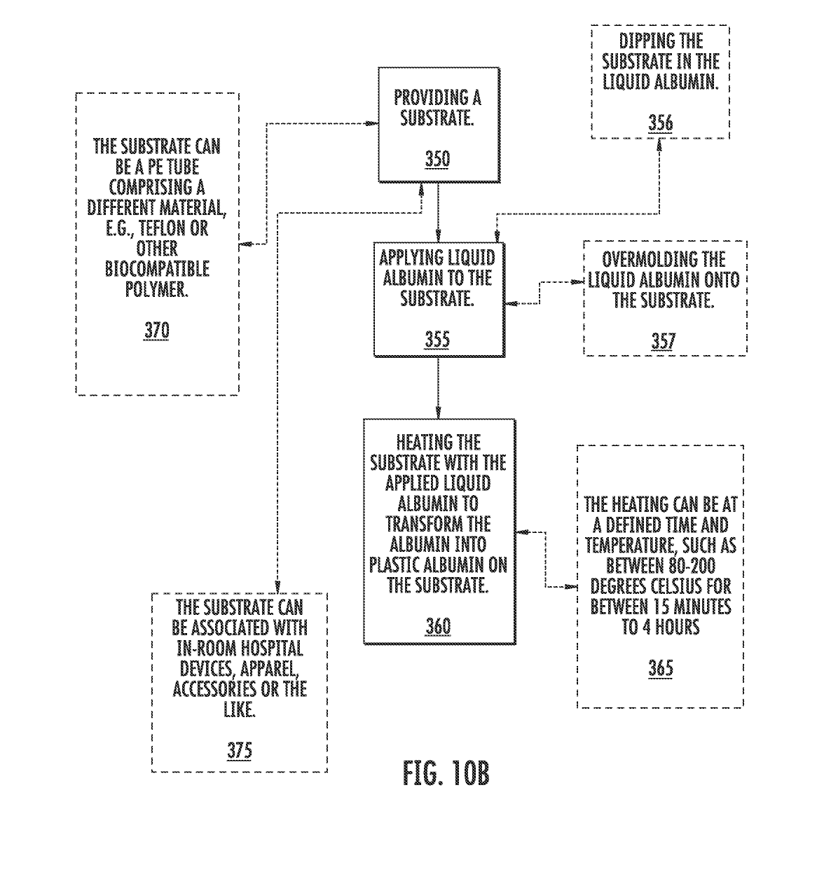

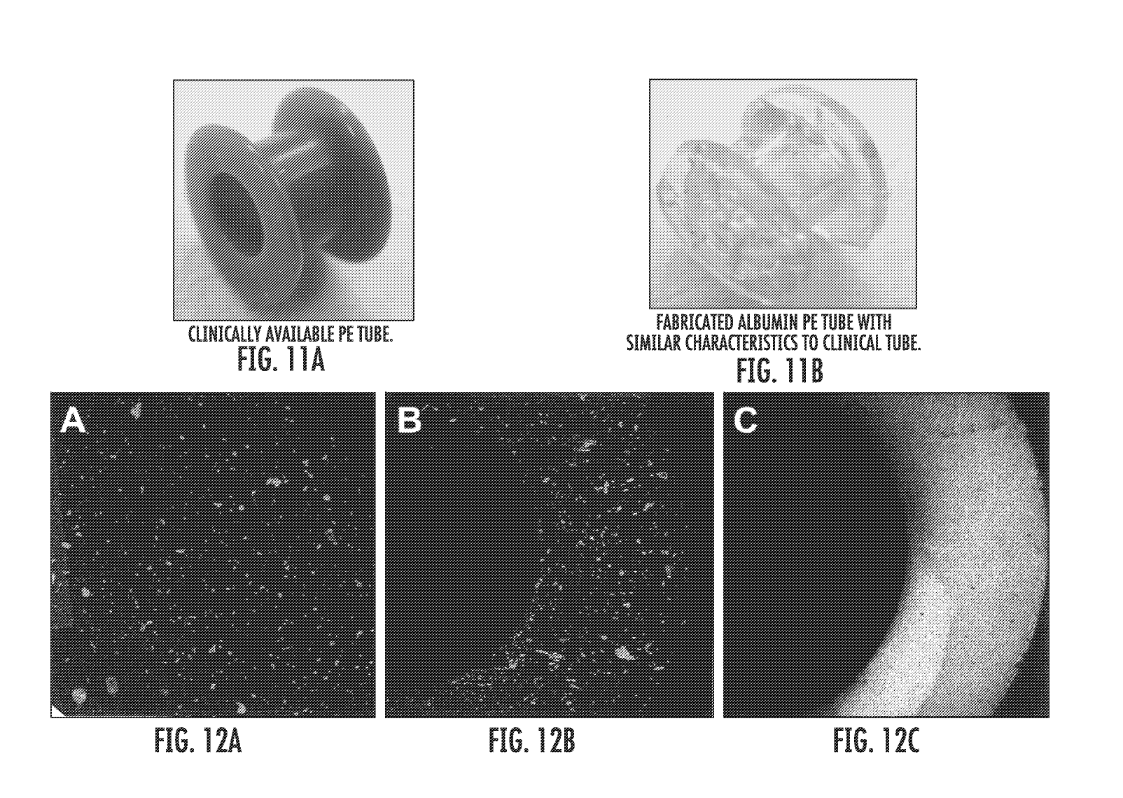

FIG. 10A illustrates exemplary operations that can be used to fabricate medical devices according to embodiments of the present invention. A liquid concentration of albumin can be provided with a concentration of albumin that is between about 5-90% (block 400). The liquid albumin solution can be placed into a mold (block 410). Then, the mold with the albumin material can be heated to a defined temperature (e.g., in an oven or autoclave at a temperature of between about 80-200 degrees Celsius) for a defined time (block 420). A solid molded (plastic) albumin body is formed (block 430).

The liquid solution can be prepared by providing dry powder albumin and dissolving it (block 402).

At least one drug can be added to the powder and/or liquid solution (block 405).

The solution can have a viscosity of about 0.0082 Pa*s (measured at room temperature) (block 411). However, it is noted that the viscosity (and viscosity below about 0.0090 can vary based on shear rate). The heating can be carried out in an autoclave using a cure temperature can be between 110-130° C. with a time of between 15 minutes to 1 hour, typically about 30 minutes (block 422).

The solid molded plastic albumin body can be a PE tube (block 432).

The albumin in the solution can have a concentration between about 25%-50%. The viscosity of the final concentrated solution (when introduced into the mold) can be as high as 51.81 Pa*s (measured at room temperature) (block 412).

One or more drugs can be applied to the solid body via immersing, coating, spraying or otherwise applying (block 434).

FIG. 10B is another flow chart illustrating exemplary operations that can be used to form plastic albumin onto target substrates. As shown, a target substrate can be provided (block 350). Liquid albumin solution can be applied to the substrate (block 355). The substrate with the applied liquid albumin can be heated to transform the liquid albumin into plastic albumin on the substrate (block 360). The heating can be at a defined time and temperature, such as between 80-200 degrees Celsius for between 15 minutes to 4 hours (block 365). The substrate can be a PE tube comprising a different (plastic or polymeric) material, e.g., TEFLON or other biocompatible polymer (block 370). The substrate can comprise any suitable material that does not melt during the curing/heating process. In some embodiments, the substrate may be a metallic-based and/or silicone-based material. The substrate can be associated with in-room hospital devices, apparel, accessories or the like (block 375).

The liquid albumin can be applied to the substrate in any suitable manner (dipped, sprayed, immersed, overmolded, poured onto). In some embodiments, the substrate is dipped in the liquid albumin (block 356). In other embodiments, the liquid albumin is overmolded onto the substrate (block 357).

According to some embodiments, an implant that includes a protein-based coating material over all or a portion of the surface of the implant may be provided. Beneficially, the coating material can be a relatively thick, robust polymeric material. Moreover, the ability of the coating material to prevent bacterial colonization and biofilm formation is not compromised with autoclave sterilization. Thus, the presence of the coating material can prevent bacteria colonization and biofilm formation on devices such as implants. Beneficially, the devices, e.g., coated implants, can exhibit enhanced biocompatibility as well as decreased likelihood of biomaterial-centered infection.

The protein-based coating material can be utilized to form a relative thick coating layer of consolidated proteins on a surface of an implant. By way of example, the coating layer can be between about 0.0001 millimeters and about 10 millimeters in thickness, for instance between about 0.5 and about 5 millimeters in thickness or between about 1 and about 4 millimeters in thickness. Accordingly, due to both the high consolidation of the proteins in the coating layer (brought about by the application of heat and pressure during formation, as described in further detail below) and the thickness of the coating layer, the coating layer may be very robust. The coated surfaces can exhibit a very slow degradation profile and can be stable in vivo for about 4 months or greater.

The mechanical and other physical properties of the coating material as well as the interfacial adhesion between the device (e.g., implant) and the coating may significantly decrease (or may even prevent) bacterial centered infection at the device (e.g., implant). Such a decrease can reduce the need for device (e.g., implant) removal and revision surgeries, thereby considerably reducing medical costs and patient suffering. Further, it is believed that the bacteria-repelling, stable coating can have enormous impact on both the military and civilian sectors of surgery and medicine.

Through selection of the protein(s) utilized in the protein-based coating material, the coated implants can be non-carcinogenic and non-allergenic and can be utilized without instigating an inflammatory response following implant. In addition, the coating material can be formed so as to be either chemically inert and, where desired, can be formed so as to be biodegradable over time. In addition, the coating material can be loaded with one or more biologically active agents (i.e., drugs) and in the aqueous environment of implantation can become gel-like, so as to allow delivery of the biologically active agents to the local environment. Moreover, formation techniques for forming and applying the coating material to the surface of the implant can be cost-effective while forming the robust coating layer over all or a portion of the surface of an implant.

The protein-based coating material can be a robust plastic that comprises protein polymers consolidated in a strong, moldable layer that can cover all or a portion of an implant. For instance, a coating material molded to form an isolated structure (i.e., comprising only the protein-based coating material and isolated from an implant to which it may be applied as a coating) can exhibit a tensile stress of from about 5 MPa to about 60 MPa, from about 15 MPa to about 50 MPa, or from about 20 MPa to about 45 MPa. The tensile strain of the isolated coating material can be from about 0.01 mm/mm (1%) to about 0.07 mm/mm (7%), from about 0.02 mm/mm (2%) to about 0.06 mm/mm (6%), or from about 0.03 mm/mm (3%) to about 0.04 mm/mm (4%). The tensile modulus of the isolated coating material can be from about 300 MPa to about 6000 MPa, for instance from about 400 MPa to about 5000 MPa, or from about 1000 MPa to about 4000 MPa. Tensile characteristics can be determined according to ASTM No. D638-86 at 20° C., 65% RH with a 1.5 mm/min cross head speed and a static 100 kN load cell. It should be understood that all ranges of the present disclosure are provided for illustrative and informative purposes but should not be considered limited to the specified ranges as they may include combinations of the values specified as well as values falling outside of these ranges.

The strength characteristics of the coating material indicate that use of the protein-based coating material can reduce the stress shielding problem on surrounding tissue that is common with many implants. For instance stress shielding problems on surrounding bone tissue are common with metal orthopedic implants, often leading to implant failure. Use of disclosed coating materials can alleviate such problems and prevent implant failure.

Proteins for use in forming the protein-based coating material can vary and can be selected so as to provide one or more desirable characteristics to the coated implant. For example, natural or synthetic proteins can be utilized to form the coating material including animal-derived proteins, plant-derived proteins, and so forth. Combinations of proteins can also be utilized in forming the coating material.

In some embodiments, one or more albumin proteins can be utilized in forming the coating material. Albumin proteins as may be utilized in forming a coating material can include, for example and without limitation, ovalbumin, serum albumins, preproalbumins, proalbumins, and so forth. In some embodiments, the coating material can include human serum albumin.

Human serum albumin is the most abundant protein in human blood plasma and is produced in the liver. It constitutes about half of the blood serum protein and is soluble and monomeric. It serves to transport hormones, fatty acids, and other compounds, buffers pH, and maintains osmotic pressure, among other functions. Human serum albumin is synthesized in the liver as preproalbumin, which has an N-terminal peptide that is removed before the nascent protein is released from the rough endoplasmic reticulum. The product, proalbumin, is in turn cleaved in the Golgi vesicles to produce the secreted albumin.

Albumin proteins such as serum albumins can possess good antibacterial activity over many bacterial strains, including S. aureus, S. epidermidis, E coli, S. mutans, S. mitis, Pseudomonas sp, Actinomyces sp, Actinobacillus sp, and Porphyromonas gingivalis. While the mechanism for this activity remains largely unknown, it is believed that serum albumin in a coating can change substratum surface hydrophobicity and provide hydration around the coated surface so as to inhibit bacterial adhesion. Specifically, hydrophobicity of the coated surface can decrease, which may diminish adhesion of hydrophobic bacteria. In addition, albumins can have a negative surface charge at the pH of blood, meaning that electrostatic repulsion between bacteria (surface charge of bacteria is mostly negative in vivo) and the coated surface can be promoted. An albumin-based coating material can also exhibit a strong binding activity, which can effectively seal off the implant surface and promote albumin characteristics at the surface of the coated implant. This can also shield active sites on the uncoated implant surface that otherwise could bind potentially damaging proteins and bacteria.

Proteins as may be utilized in forming the coating material can be utilized in the native state or can be processed prior to formation of the coating material. As is clear to one skilled in the art, the average size and distribution of sizes of the proteins in the protein-based coating material are amongst the factors that define the physical properties of the coating formed from the material. As such, in some embodiments, it may be advantageous to denature the proteins of the coating material so as to better control the characteristics of the coating formed of the protein-based coating material. Additionally, many inexpensive protein sources include a mixture of different types of proteins as well as a proportion of carbohydrates, some of which may not be desired for use in the coating material. Denaturation of the proteins in such a mixed proteinaceous source material can be utilized to separate the desired proteins (e.g., the albumin proteins) from other, less desirable proteins and carbohydrates present in the source material.

Denaturation of the proteins for the coating material can be carried out according to any method as is generally known in the art. Denaturation of proteins involves the disruption and possible destruction of both the secondary and tertiary structures. Since denaturation reactions are not strong enough to break the peptide bonds, the primary structure remains the same after a denaturation process. Denaturation disrupts the normal alpha-helix and beta sheets in a protein and uncoils it into a random shape. According to some embodiments, one or more denaturation processes can be carried out so as to disrupt hydrogen bonds, salt bridges, disulfide bonds, and/or non-polar hydrophobic interactions, of the protein(s) utilized in forming the coating material.

According to some embodiments, heat can be used to denature the protein(s) through disruption of hydrogen bonds and/or non-polar hydrophobic interactions. While the temperature for denaturation of particular proteins can vary somewhat, in general, a heating process can denature the proteins without destruction of the peptide bonds when carried out at a temperature of between about 40° C. and about 180° C., for instance between about 60° C. and about 130° C. By way of example, human serum albumin begins to denature at about 86° C.

An alcohol wash can be utilized to denature the proteins of the coating material. For instance, a 70% alcohol solution can denature the proteins by disrupting the side chain intramolecular hydrogen bonding. Other denaturation processes can include, without limitation, disruption of salt bridges through use of acids and bases, heavy metal salts, and so forth. The use of heavy metal salts may also disrupt disulfide bonds due to their high affinity and attraction for sulfur. Reducing agents can also be utilized to disrupt disulfide bonds of a protein according to standard practice. One or more such processes can be carried out to partially or completely denature proteins of the coating material.

The coating material can include additional materials in conjunction with the one or more proteins. For instance, in some embodiments, the coating material can include additional polymers in a polymeric blend with the protein(s). Polymers as may be blended with the protein can include synthetic or natural polymers, as desired. A second polymer of the coating material can be selected so as to provide desirable characteristics to the coating material. In general, a polymer blend can include greater than about 1% by volume of one or more proteins, for instance greater than about 5%, greater than about 15%, greater than about 25%, greater than about 50%, greater than about 75%, greater than about 80%, or greater than about 90% by volume of one or more proteins in conjunction with one or more non-proteinaceous polymers.

In some embodiments, a biodegradable implant can be coated with the coating material. In these embodiments, the coating material can also be biodegradable. Thus, when considering a coating material including a polymer blend, the coating material can incorporate one or more implantable, biodegradable polymers in conjunction with the protein of the coating material. Examples of such polymers for formation of an implantable, biodegradable coating material can include, without limitation, polysaccharides (e.g., starch, cellulose, hyaluronic acid, etc.), polyesters (e.g., poly(hydroxyalkanoates), poly(lactic acid), etc.), copolymers (e.g., poly(lactide-co-glycolide)), polyanhydrides, etc.

Polymer blends of a coating material can incorporate non-degradable polymeric components. For instance, a coating material can incorporate a polymer as is typically utilized in forming implants, such as, without limitation, polyamides (e.g., Nylon®), silicon-based polymers (e.g., polydimethyl siloxane, silicone rubbers, etc.), polyacetals, polyalkenes (e.g., polyethylene including ultra-high molecular weight polyethylene, polytetrafluroethylene, polyethylene terephthalate, etc.; polypropylene; etc.), polyurethanes, polyesters, poly(vinyl chloride), polyacrylates (polymethacrylate, polymethylmethacrylate, etc.), polyetheretherketones, polysulfones, and so forth. In some embodiments in which a polymeric implant is to be coated with the coating material, the coating material can include a polymer as is found in the bulk implant. For example, a coating material intended for use for coating an implant formed of an ultra-high molecular weight polyethylene can incorporate an amount of an ultra-high molecular weight polyethylene. This can improve adhesion between the bulk implant and the coating material.

The protein-based coating composition can include one or more additives as are generally known in the art. Additives can include, for example, inorganic compounds such as calcium phosphate, sodium bicarbonate, calcium di-hydrogen phosphate, calcium hydrogen phosphate, calcium phosphate and calcium carbonate. Other additives can include, without limitation, plasticizers, preservatives, colorants, antimicrobial compounds (e.g., silver nanoparticles, other metal cations, and antibiotics), mold release agents, extenders, antioxidants, and so forth. In general, an additive can be present in the coating material in standard amounts, for example in an amount of up to about 70% by weight of the coating material. For instance, an additive can be present in the coating material in an amount of from about 0.5% to about 20% by weight of the coating material, for instance from about 1% to about 10% by weight.

The protein-based coating material can be utilized to coat any substrate and/or implant formed of any suitable material, such as a biocompatible, implantable material for implants. For instance, and without limitation, the protein-based coating material can be utilized to form a coating on an implant such as bone implants, catheters, cannuale, guide wires, stents, shunts, vascular grafts, heart valves, heart and ventricular assist devices, oxygenators, dialyzers, joint replacement components, sutures, tracheal tubes, artificial blood vessels, gastrointestinal segments, facial prostheses, dialysis devices, and medical instruments, and other substrates, such as furniture, computer keyboards, phone, etc.

Unless indicated to the contrary, in describing exemplary embodiments, implants and other substrates may be used interchangeably. Thus, coatings that are described in regard to implants by way of discussion herein may also be used as coatings for other substrates and vice versa.

Materials as may be coated with the protein-based coating material can include polymeric materials (e.g., polyamides, silicon polymers, rubbers, polyesters, polyethylenes, polymethylmethacrylates, etc), metals (e.g., titanium and alloys thereof, cobalt/chromium alloys, gold, silver, platinum, stainless steels, etc.), inorganics (e.g., ceramics such as alumina, zirconia, calcium phosphates including hydroxyapatites, etc), composites, e.g., carbon fiber reinforced composites, fiber reinforced bone grafts, etc.), and so forth.

In order to facilitate adhesion between the implant or other substrate surface and the coating material, the implant or other substrate surface may be pretreated according to processes as are generally known in the art. For example, the implant surface can be treated according to plasma treatment, corona discharge, and so forth.

In some embodiments, an anchoring layer can be applied to the implant or other substrate surface that can improve adhesion between the protein-based coating layer and the implant or other substrate. For example, in some embodiments an anchoring layer as described in U.S. Pat. No. 7,026,014 to Luzinov, et al., which is incorporated herein by reference, can be formed on the implant surface prior to addition of the protein-based coating layer. Briefly, according to this process, polymer surfaces are initially treated with a strong base or air corona/plasma to produce surface functional groups, such as —OH and —COOH at the surface. Following, the surface can be modified with an anchoring epoxy-containing polymer such as poly(glycidyl methacrylate) (PGMA) in order to activate the surface with reactive epoxy groups. A portion of the epoxy groups on the polymer can react at the surface binding the polymer to the surface at multiple points along the polymer. For instance, in some embodiments, the polymer can be covalently bound to the substrate surface at multiple points via epoxy groups on the polymer chain. In addition, the polymer can cross-linked with itself via other epoxy groups of the polymer. Following surface binding and cross linking, the polymer can include unreacted epoxy groups that can be available for binding to components of the coating material during formation of the coating layer.

Following any surface modification of an implant or other substrate, the coating material can be applied to the surface so as to form a robust protein-based coating on the surface. Methods for forming the coating material and applying the coating material to a surface of an implant can include both wet and dry processes.

According to some embodiments, dry blending of components of the coating layer can be carried out followed by molding of the dry blended components under heat and pressure to form the coating material. In addition, the coating material can be formed either directly onto the surface of the implant or other substrate or, alternatively, can be first formed as an isolated structure, e.g., a consolidated film of the protein-based coating material followed by adhesion of the film to the surface of the implant or other substrate. According to a dry formation process, a flowable powder of the protein(s) of the coating material can be produced for use in a subsequent molding process. If desired, the powder may be mixed with additional components of the coating material such as secondary polymers, additives, etc. In some embodiments, the particulate protein powder may have a grain size distribution ranging from about 5 microns to about 6000 microns, for instance from about 100 microns to about 400 microns.

Following mixing of the dry components, the components can be molded to form a consolidated protein-based coating material according to typical polymer processing methodology. For example, in some embodiments, the protein powders or their blends can be processed to form a film that can be applied to a surface of an implant or other substrate. By way of example, a dry protein powder can be processed according to polymer processing methodology as is generally known in the art including, without limitation, melt processing that can include calendaring, compression molding, transfer molding, extrusion and injection molding, etc. Formation of a film formed of the coating material can be produced from a polymeric melt of the dry particulate mixture through one or more of calendaring, extruding, or limiting length in compression molding.

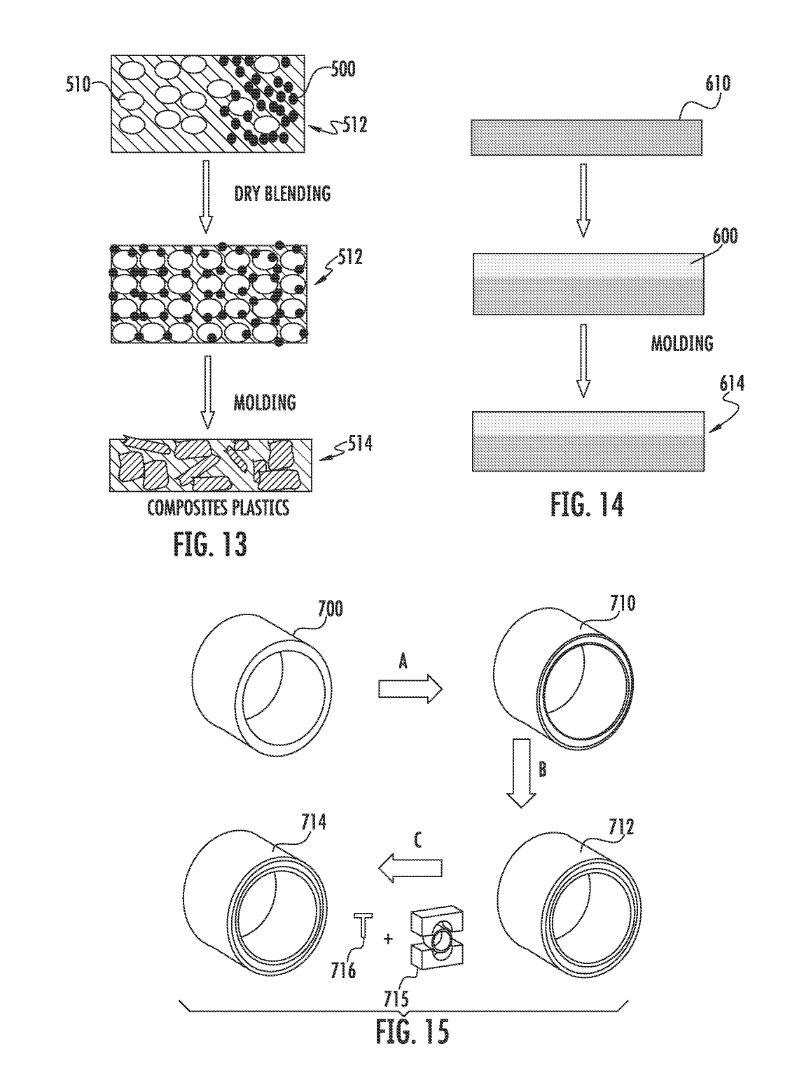

FIG. 13 illustrates one method of forming a coating material according to a dry formation technique. As can be seen, a protein powder 500 can be mixed with additional components of a coating material 510 such as, e.g., a secondary polymer, an inorganic additive, plasticizers, etc. to form a dry mixture 512. Following dry blending, the dry mixture 512 can be molded, e.g., compression molded to form a composite plastic film 514. In general, molding of the dry mixture can be carried out under heat and pressure, for instance at a temperature of from about 200° F. to about 250° F. and under a pressure of from about 20 MPa to about 40 MPa so as to consolidate the proteins of the mixture and form the protein-based coating material.

By way of example, the dry mixture can be molded according to a compression molding process. Compression molding is a method of molding in which the molding material, generally preheated, is first placed in an open, heated mold cavity. The mixture may be loaded into the mold either in the dry form of pellets or powder, or the mold may be loaded from a plasticating extruder. The mold is closed with a top force or plug member, pressure is applied to force the material into contact with all mold areas, while heat and pressure are maintained until the molding material is heated above the melting point, formed and cooled. The more evenly the feed material is distributed over the mold surface, the less flow orientation occurs during the compression stage.

Wet processing techniques for forming the protein-based coating material can include formation of a liquid including the proteins of the coating material and any desired additives of the material. The liquid can then be processed according to standard methodology to form the coating material. The protein-containing liquid can be a solution, a suspension, a colloid, or a dispersion, generally depending upon the nature of the coating material components in conjunction with the nature of the liquid carrier. In addition, the liquid can include different components in different states. For example, one or more components of the liquid may be dissolved in the carrier solvent, while other components may be present as suspended particles or colloids. As with the dry formation processes, wet processing formation techniques can be used to first form a film of the coating material followed by adhesion of the film to the surface of an implant or alternatively to directly form the consolidated coating layer on the implant surface in a one-step formation process. By way of example, thin films of the coating material can be cast from a polymer solution in a volatile solvent, a dispersion in a latent solvent, a paste including a plasticizer, or a water-based polymeric system (e.g., latexes, dispersions, or suspensions).

A thin film of the protein-based coating material can be adhered to the implant surface by use of thermal adhesion, use of an adhesive agent, a combination of application of heat and pressure, radiation treatment (e.g., application of microwave radiation), or the like. According to some embodiments, a combination of heat and pressure can be used, which can improve conformation of the thin film to the surface of the implant and can provide robust adhesion between the coating layer and the implant surface. As previously mentioned, the surface of the implant or other substrate may also be pretreated so as to improve interfacial adhesion between the coating material and the implant surface.

FIG. 14 illustrates a process in which the protein-based coating material can be formed directly on the surface of an implant or other substrate. According to this embodiment, a mixture 600 that includes the components of the protein-based coating material can be applied to the implant or other substrate surface 610. The mixture can be either a dry or wet mixture, as desired. Following application of the coating material to the implant surface, heat and pressure can be applied as described above so as to consolidate the components of the coating material and adhere the coating material to the surface of the implant or other substrate and form the coated device 614.

A liquid mixture that includes the components of the coating material can be applied to the implant surface according to any suitable methodology including, without limitation, dip coating, spray coating, spin coating, knife coating, and the like. The viscosity of the liquid mixture can be adjusted as necessary so as to coat the implant surface with the coating material prior to subjection of the liquid to heat and pressure to form the consolidated coating layer. In general, the viscosity of the liquid may be adjusted by alteration of the relative amount of the carrier liquid, as is known.

As discussed above, the protein(s) of the coating material can be denatured prior to consolidation of the coating material. In some embodiments, denaturing can take place following application of the liquid to the implant surface and prior to consolidation of the coating material. For example, the liquid containing the components of the coating material may be applied to the implant or other substrate surface and following application, the implant may be heat treated to a temperature of greater than about 41° C. so as to denature the proteins of the coating material. Following the heat treatment, the implant may be subjected to heat and pressure to consolidate the coating material and adhere the coating to the implant surface.

FIG. 15 illustrates another formation process as may be utilized to form a coated implant or other substrate. In this embodiment, a tubular implant 700 can be surface treated at “A” with an epoxy-containing polymer, e.g., poly(glycidal methacrylate) so as to improve adhesion between the coating material and the implant or other substrate surface. Deposition of the polymer can form a primary ultrathin anchoring layer 710 on the implant or other substrate. Following formation of the anchoring layer 710, a mixture 712 that includes the components of the coating material can be applied to the anchoring layer at “B”. As described above, the mixture can be a dry mixture or a liquid mixture, as desired. Heat and pressure “T” 716 can be applied to the mixture, for instance by use of molding elements 715 so as to consolidate the mixture and form the protein-based coating layer 714 on the surface of the implant.

As mentioned, following consolidation the protein-based coating may swell and form a flexible gel in an aqueous solution and may return to the original state after drying. This characteristic can facilitate loading of a biologically active agent into the coating layer prior to implant. For example, following coating of the material onto the surface of an implant or other substrate, the coated device can be submerged in an aqueous solution containing one or more biologically active agents. Upon swelling and gelling of the coating material, the biologically active agents can diffuse into the coating material along the concentration gradient. Following implantation, the process can be reversed, and the biologically active agents can be delivered to an implant site. The gel-like state of the coating material can also be beneficial following implantation due to the nature of the coating material itself. For instance, following implantation, the hydrated environment of the swollen gel-like coating material may reduce thrombogenity of the implant.

Biologically active agents as may be incorporated in a coating can include proteinaceous agents, small molecules, antibiotics, antithromobogenic factors, or any other compound as may be beneficially released from the implant surface. By way of example, and without limitation, growth factors may be incorporated in a coating material including, but without limitation to, endothelial cell growth factor, epithelial growth factor, bone morphogenic proteins, fibroblast growth factor, platelet derived growth factor, hepatocyte growth factor, nerve growth factor, etc. Other biologically active agents as may be incorporated in a coating material can include, for example, an antimicrobial agent such as lysosyme or penicillin; an antithrombogenic agent such as heparin, albumin, streptokinase, tissue plasminogin activator (TPA) or urokinase; a thrombogenic agent such as collagen or a hydrophilic polymer such as polyethylene glycol (a synthetic polymer), chitosan or methyl cellulose, and other proteins, carbohydrates and fatty acids. Of course, combinations of biologically active agents may also be incorporated in the coating material.

According to some embodiments, bone morphogenic proteins (BMPs) including both cytokines and metabologens, may be incorporated into the coating layer to improve bone healing as well as possibly partially remedy infected bone defects. BMPs interact with specific receptors on the cell surface (bone morphogenetic protein receptors (BMPRs)). Signal transduction through BMPRs results in mobilization of members of the SMAD family of proteins. The signaling pathways involving BMPs, BMPRs and SMADs are important in the development of the heart, central nervous system, and cartilage, as well as post-natal bone development. BMPs have an important role during embryonic development on the embryonic patterning and early skeletal formation.

The invention will now be described in more detail in the following non-limiting examples.

### EXAMPLES

### Example 1

FIG. 11B illustrates a novel albumin pressure equalization tube for the treatment of otitis media.

The PE tubes were formed using human serum albumin. The human albumin plastic PE tubes were made with similar characteristics, such as size and shape, to currently available PE tubes (see FIG. 11A). A hollow biodegradable albumin plastic tube was molded in order to prevent the attachment of bacteria and the formation of biofilm. The PE tube can facilitate the drainage of the middle ear cavity similar to current clinically available tubes. Staphylococcus aureus bacteria was cultured in a tryptic soy broth and resuspended in PBS. The fabricated albumin PE tubes were immersed into the bacterial suspension and cultured for a period of 3 hours to allow initial adhesion and replication. Then, the tubes were stained using a LIVE/DEAD assay and imaged using a scanner laser confocal microscope. Albumin PE tubes were compared to clinically available PE tubes to determine if either had an effect on preventing bacterial adhesions.

Human albumin plastic PE tubes were successfully fabricated with similar characteristics to current PE tubes used clinically. In vitro bacteria culture was used to determine the effectiveness of the albumin plastic to prevent bacterial adhesions and colonization. After 3 hours in culture, there were abundant live bacteria attached and colonized on the commercial PE tubes (FIG. 12B). In contrast, there was no live bacteria on the albumin plastic based PE tubes (FIG. 12C). A positive control (FIG. 12A) consisted of bacteria grown on a glass coverslip to demonstrate the assay was working properly. It was determined that the albumin tubes (FIG. 11B) will prevent bacterial adhesions while also preventing biofilm formation when compared to current tubes clinically available.

Biofilm formation due to bacterial contamination of PE tubes is a major problem associated with middle ear infections. Otolaryngologic diseases can arise from the biofilm formation, introducing a variety of implant associated conditions related to PE tube infection. An albumin plastic PE tube should circumvent many of the problems currently encountered by children with PE tubes today by preventing bacterial biofilm formation.

### Example 2

A consolidated plastic was prepared of human serum albumin. The solution of Human serum albumin (HSA) comprised 25% HSA in phosphate buffer saline (PBS). After freeze drying, the HSA (molecular weight˜66,500 D) contained approximately 4% bonded water.

Following formation, a variety of physical characteristics were determined for the material.

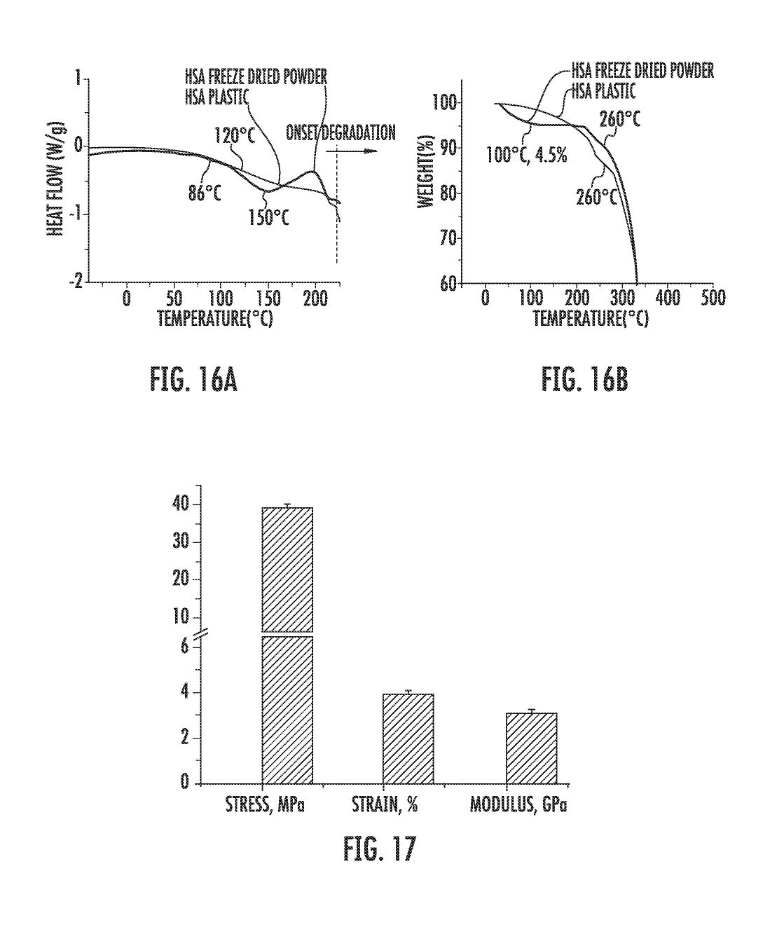

FIG. 16A shows Differential Scanning calorimetry (DSC) (and FIG. 16B shows Thermogravimetric Analysis (TGA) of both the protein powder and the consolidated plastic prepared from the powder. Human serum albumin starts denaturing at a temperature of 86° C., however the original endothermic peak due to the denaturation (150° C.) was not detected for the plastic samples obtained (FIG. 16A). The result may indicate that another type of folded structure is formed during the plastic preparation. TGA results showed that a different weight loss pattern was observed for the plastic samples (FIG. 16B) in comparison to the original powder. Specifically, the first water weight loss occurred over a more extended temperature range: from room temperature to approximately 250° C. The slowdown of the water loss can be explained by the denser structure of the plastic sample as compared with the protein powder. The onset temperature of degradation (260° C.), however, was virtually unaffected by the compression molding. Thermal analysis revealed temperature window for effective and safe (no degradation) thermal treatment of the protein films. It appeared to be within a range of 90° C. to 120° C.

Tensile characteristics for the consolidated plastic were determined according to ASTM No. D638-86 at 20° C., 65% RH with a 1.5 mm/min cross head speed and a static 100 kN load cell. Results are shown in FIG. 17. As can be seen, the consolidated protein material exhibits excellent tensile stress, strain, and modulus characteristics for use in conjunction with an implant.

### Example 3

To analyze how albumin will influence bacterial adhesion when it is dry blended with a secondary polymer, samples were formed utilizing ovalbumin and ultra-high molecular weight polyethylene (UHMWPE), which was chosen due to its common utilization in forming implants. Samples including 100% UHMWPE, 100% ovalbumin, and various blends of ovalbumin with UHMWPE were prepared with compression molding techniques as shown in FIG. 13 followed by cleaning with DI water, 70% water-EtOH, and EtOH to ensure that samples were free of contaminants and pathogens.

Mechanical properties obtained from tensile measurements are presented in Table 1, below. Blends are reported by weight percent.

TABLE 1

| | | | Tensile |
|---|---|---|---|
| | | | Strain |
| | Modulus | Tensile Stress | (mm/mm) |
| 100% UHMWPE | 415.755 | 12.76808 | 0.050264 |
| 5% ovalbumin | 441.9267 | 13.58668 | 0.050516 |
| 10% ovalbumin | 482.47 | 16.19771 | 0.053628 |
| 15% ovalbumin | 545.3275 | 15.3122 | 0.048295 |
| 20% ovalbumin | 686.12 | 22.59 | 0.05177 |
| 100% ovalbumin | 1606.983 | 44.2683 | 0.04705 |

Tensile stress and strain were calculated at 2% offset strain point. Initial modulus was calculated as initial slope. At least 6 measurement were done and averages were calculated

In order to determine the level of adhesion between components in the composite, the Kerner (good adhesion and bad adhesion model) model was employed (FIG. 18). The model showed good adhesion between particles at each loading. This indicates that the good mixing of components in the dry state was maintained during processing. Upon denaturation, amino acid side groups would change their conformation to express non polar segments that can interact with the environment (hydrophobic UHMWPE). This consequence of conformational change may improve adhesion.

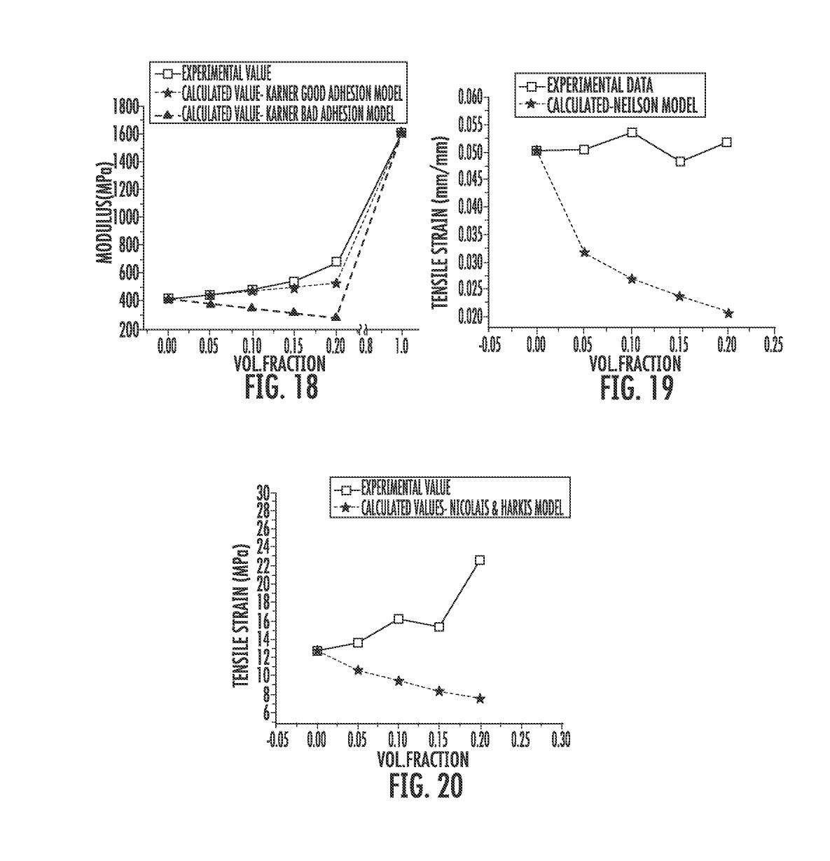

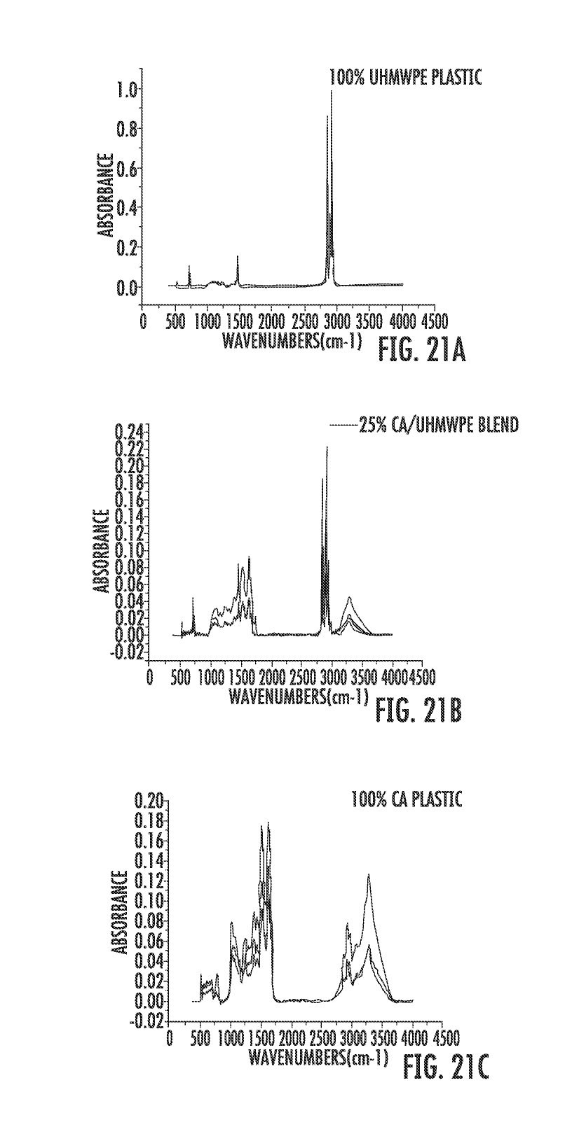

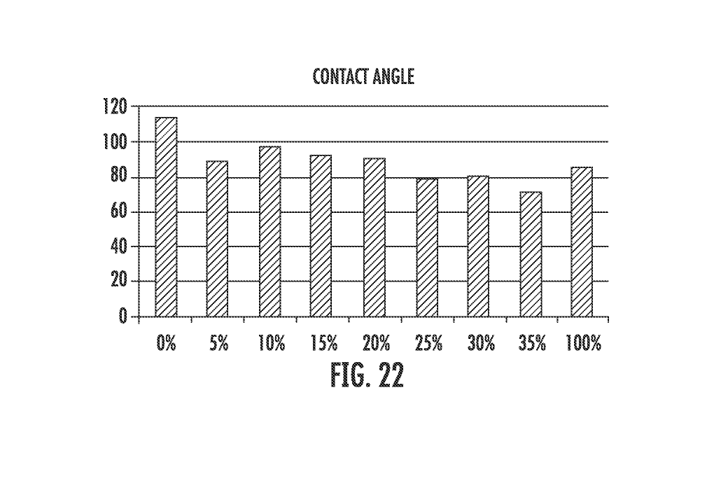

Experimental strain results were compared with the Neilsen model calculation. The comparison indicates that albumin does not have any significant effect on extensibility of structures (FIG. 19). The experimental strain values were found to be relatively similar, but higher than the Neilson model calculation, which assumes good adhesion. Stress values are modeled with the Nicolais-Narkis approach and were found to be greater than the calculated values (FIG. 20). The Nicolas model assumes no adhesion between the filler and matrix. Increased albumin content increases stress.

The mechanical analysis proved the existence of adhesion between the two polymer components, which is in good agreement with well-known models.

Surface morphology and chemistry were analyzed with scanning electron microscopy and Fourier Transform Infrared spectroscopy. A total of ten analyses were performed for one sample and different absorbance ratios were found. FTIR results (FIG. 21) indicate the presence of albumin on the surface, which changes with the ratio of albumin concentration. FIG. 21A is 100% UHMWPE, FIG. 21B is a 25:75 ovalbumin/UHMWPE blend, and FIG. 21C is 100% ovalbumin material. However, different absorbance ratios in the same sample were also found, as shown. This could be related to nonhomogenous distribution.

Wetting behavior of blends was also examined including static contact angle measurements. Static contact angles of three different samples for each material were measured at room temperature by the sessile drop method using water. At least three measurements from a different region of each surface were conducted. No direct relation was observed between the concentration of protein and wetting behavior. Results (FIG. 22) point out the heterogenic distribution of albumin in blends, which confirmed the FTIR results (FIG. 21). FTIR results together with contact angle measurements could be related with homogeneity of blending and the influence of compression molding.

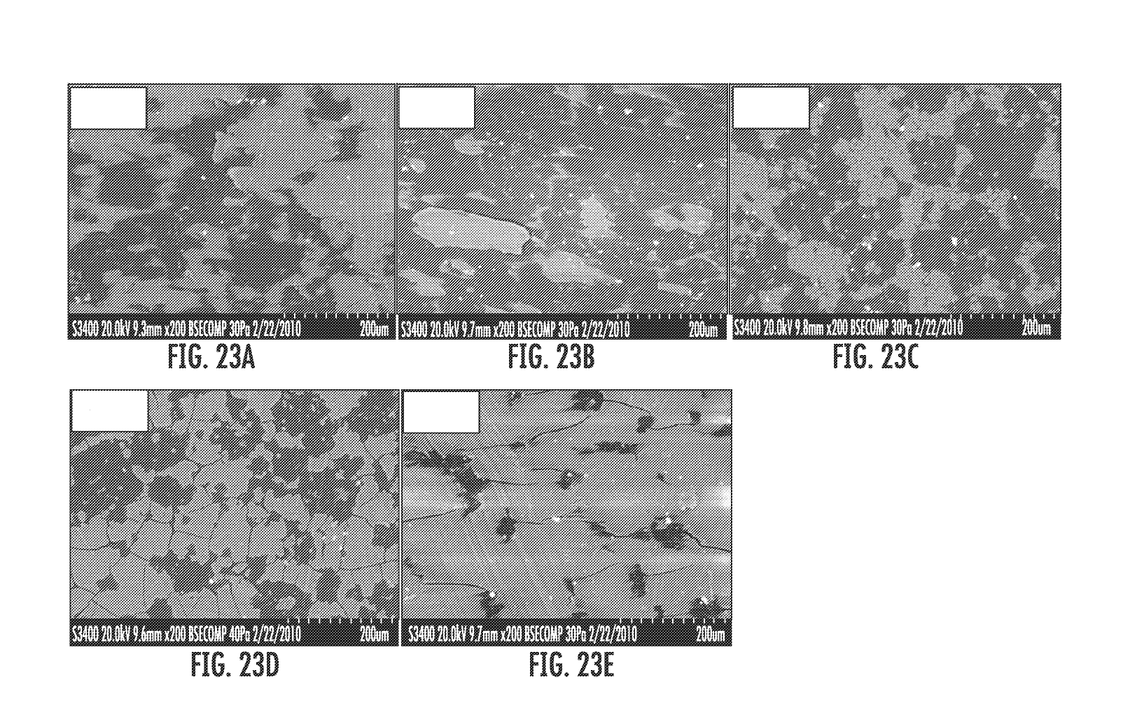

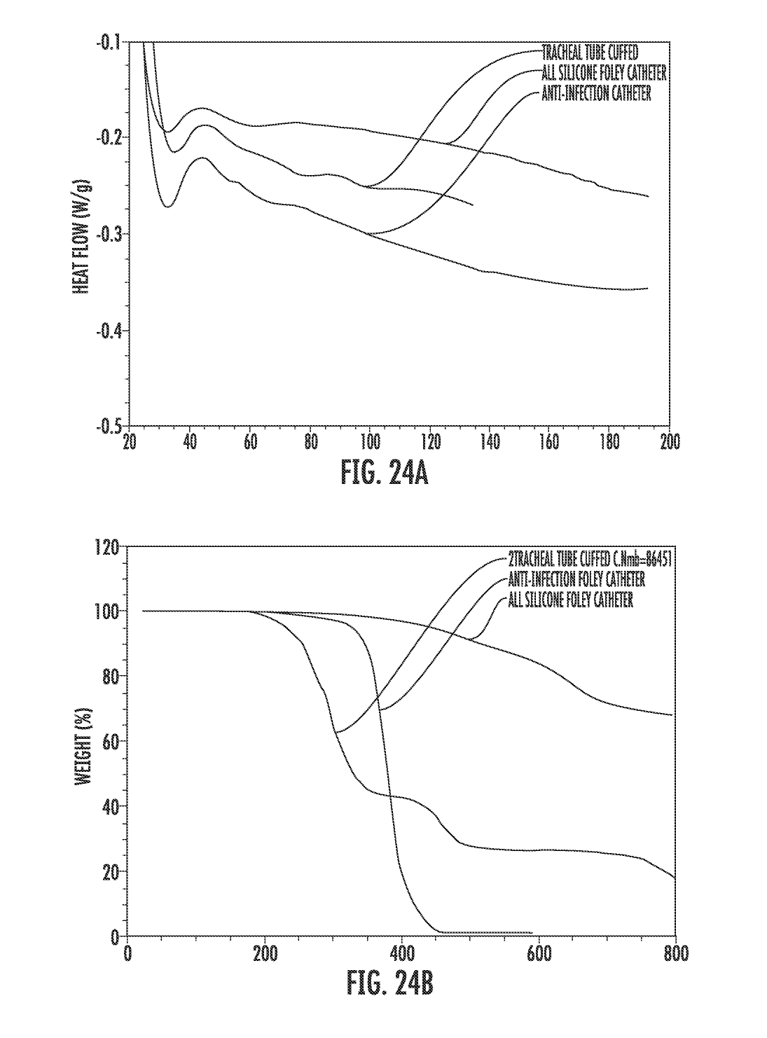

SEM images (FIG. 23) show phase separation of albumin and UHMWPE particles in the blend for several samples including a 15:85 ovalubumin/UHMWPE blend (FIG. 23A), a 25:75 ovalbumin/UHMWPE blend (FIG. 23B), a 35:65 ovalbumin/UHMPE blend (FIG. 23C), a 45:55 ovalbumin/UHMWPE blend (FIG. 23D), and a 55:45 ovalbumin/UHMWPE blend (FIG. 23E).

To examine the ability of the protein-based coating materials to prevent bacterial adhesion, a bacterial suspension was prepared from a S. aureus stock plate. Bacteria were placed in 5 ml of tryptic soy broth, and incubated in a test tube for 18 hrs at 37° C. with agitation. Subsequently, a 1 ml aliquot of broth was centrifuged for 5 minutes at 1200 rpm, and then the supernatant was aspirated. The pellet of bacteria was re-suspended in 1 ml sterilized PBS and later added to 49 ml of PBS. Bacteria were stained with SYTO 63 at room temperature in the dark for 30 minutes and subsequently centrifuged and resuspended several times to remove excess dye.

Plastic samples were immersed in 5 ml of the bacterial suspension (2×10⁷ bacteria/ml). All samples were then incubated at 37° C. for 2 hours, followed by gentle rinsing with sterile PBS three times and air drying. One sample for each group was not washed to use as a control sample. This unwashed sample was used to examine how bacteria appeared on the surface of albumin. This was utilized to show that if bacteria attached to the albumin, they would be clearly distinguishable even with a background of albumin.

A Nikon confocal fluorescence microscope was used to analyze adhesion of bacteria. At least three images were taken of each sample to get a reliable average. Bacteria attachment to the UHMWPE surface was greater than to albumin and to an albumin-containing blend surface. Results indicated that the albumin prevented bacterial attachment to the surface.

### Example 4

To examine the capability of typical implant devices to withstand the heat and pressure necessary to form a consolidated coating on the surface, several devices include a tracheal tube cuffed, an all silicone Foley catheter, and an anti-infection Foley catheter were examined. To verify that the devices can withstand protein film formation temperatures, differential scanning calorimetry and thermogravimetric analysis were carried out for the items. Results are shown in FIG. 24. TGA analysis (FIG. 24B) revealed that all three materials start to degrade at the temperatures close or higher than 200° C. Degrading behavior of the chosen materials would allow for preparing protein films on the surface without causing thermal degradation of the materials. Moreover for all three catheters tested, DSC measurements (FIG. 24A) do not show major thermal transitions in the range from ambient temperature to 140° C. indicating an absence of substantial change in mechanical properties and shape within this temperature range thus allowing safe protein film formation.

### Example 5

Titanium substrates were treated according to several different combinations of processes to form coatings on the surfaces. Processing included the following:
Abrading surface with 40 grit aluminum oxide sand paper
Ultrasonic cleaning with multiple solvents including DI water, acetone, and methylethyl ketone −30 minutes each cleaning
Formation of a poly(glycidyl methacrylate) anchoring layer
   Plasma treatment (30 min.)
   Water soaking for 10 min. followed by drying in 80° C. oven
   %0.5 PGMA/MEK was used
   Samples were soaked in PGMA solution for 15 min. No annealing was done after PGMA coating
Human serum albumin (HSA) coating
   %10 HSA aqueous solution was used
   Samples were dipped into the albumin solution for 30 min. and then solution was pumped out.
Annealing treatment in a 155° C. vacuum oven for 1 hour
Final cleaning with several solvents
   Hexane, toluene, acetone, ethanol/water(70/30), ethanol: samples were soaked in solvent for 30, two times for each solvents

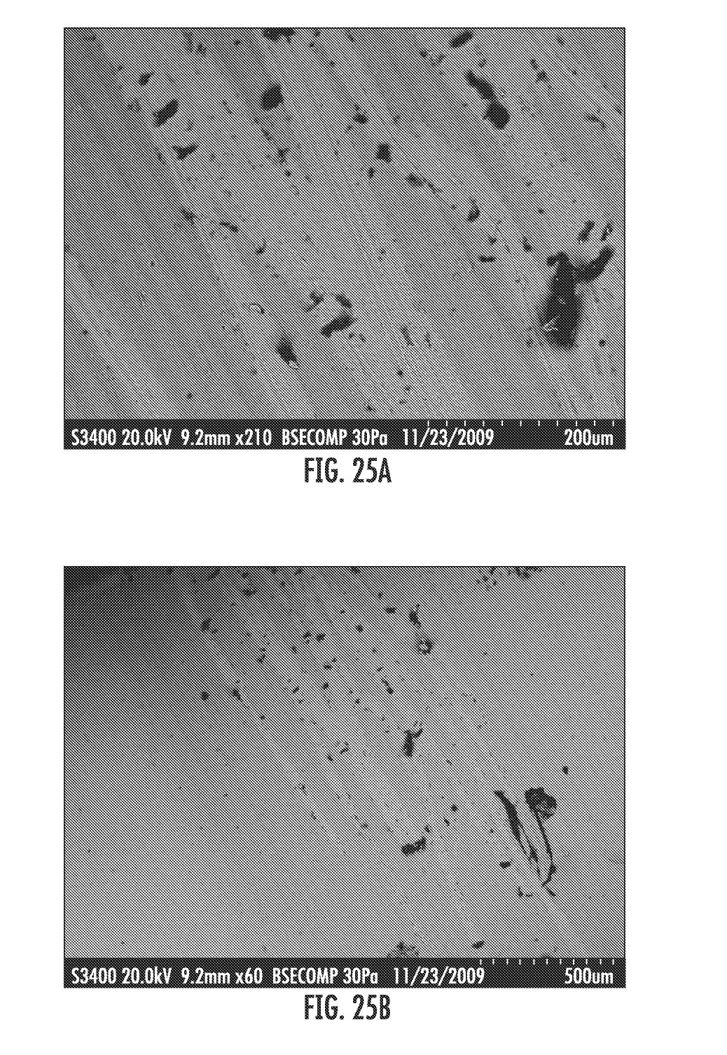

FIGS. 25A and 25B illustrate a sample at two different magnifications that was not abraded, and was cleaned according to the ultrasonic treatment process followed by formation of the PGMA coating layer. The SEM images show the PGMA coating on the scratched titanium surface.

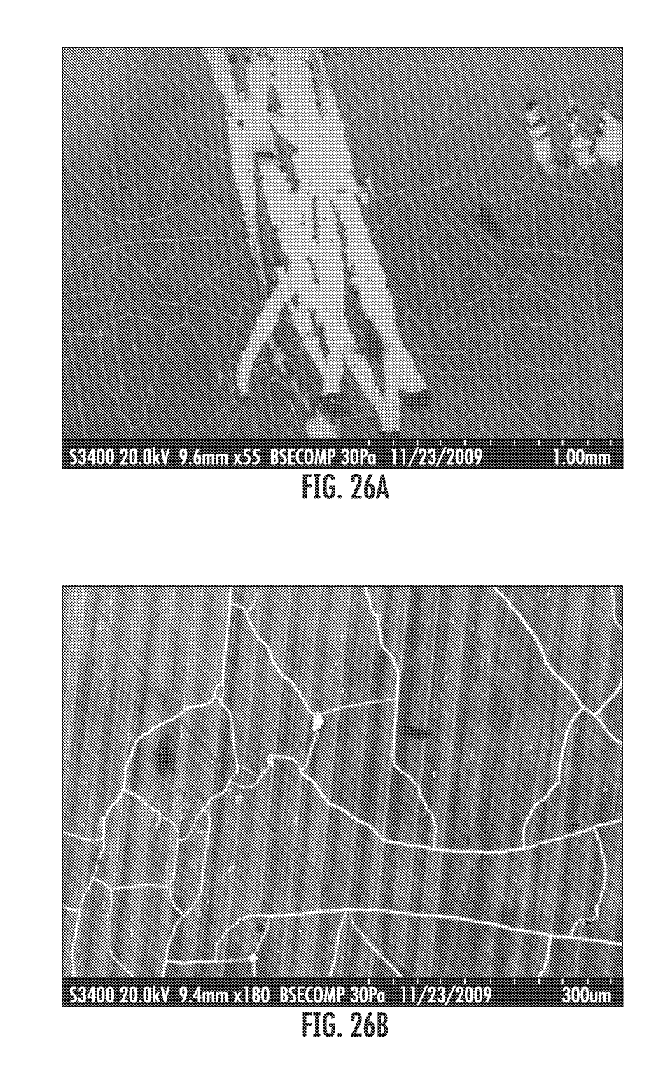

FIGS. 26A and 26B illustrate a sample at two different magnifications that was not abraded, and was cleaned according to the ultrasonic treatment process followed by formation of the PGMA coating layer and application of the HSA solution. The HSA solution was then dried at room temperature. Based upon a similar process on a silicon wafer coating, the HSA solution was estimated to be coated at a thickness of about 300 nm. The SEM images show the thick coating on the surface via the scratches seen on FIG. 26A and the cracks in the dried coating on FIG. 26B.

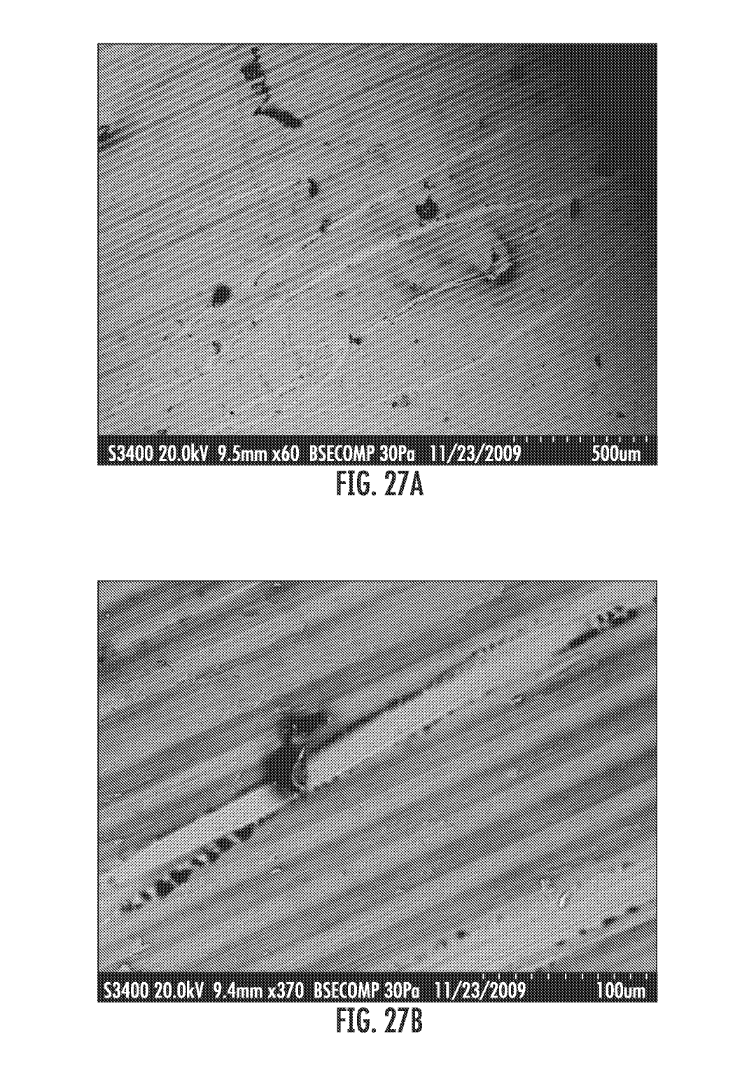

FIGS. 27A and 27B illustrate a sample at two different magnifications that was not abraded, and was cleaned according to the ultrasonic treatment process followed by formation of the PGMA coating layer and application of the HSA solution. The HSA solution was then annealed as described above and a final wash was carried out. Based upon a similar process on a silicon wafer coating, the HSA solution was estimated to be coated at a thickness of about 300 nm, but the coating was estimated to be thinner than the coating formed on the non-annealed sample shown in FIG. 26.

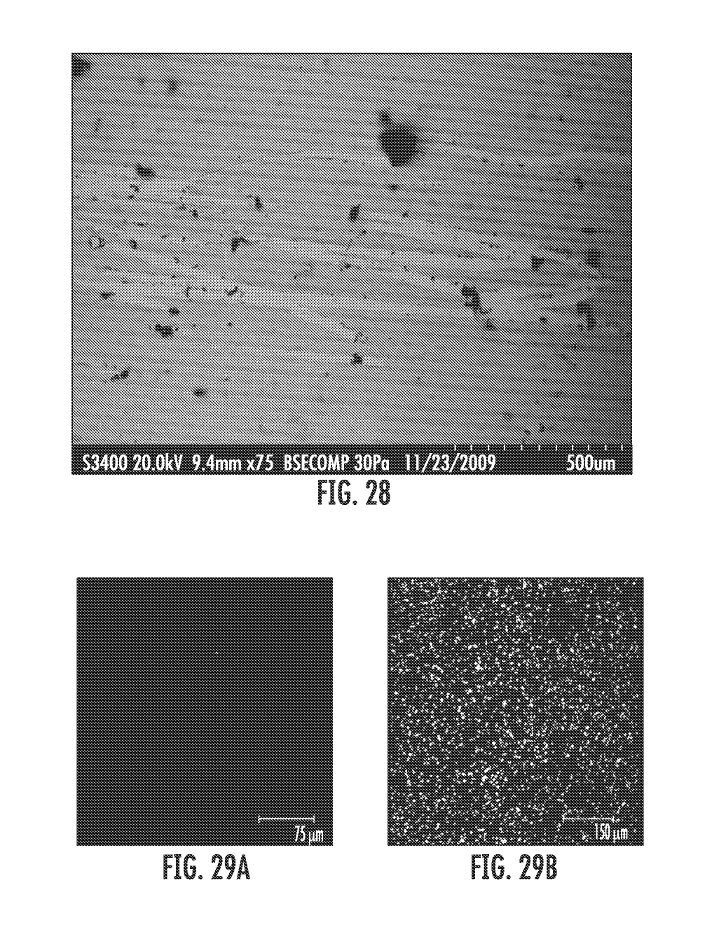

FIG. 28 illustrates a sample that was not abraded, and was cleaned according to the ultrasonic treatment process followed by formation of the PGMA coating layer and application of the HSA solution. The HSA solution was then annealed as described above and compression molded followed by the final wash.

Coating layers were also formed on titanium substrates using an HSA solution including 2% by weight rhodamine as a label. The same procedures as described above were used including both abraded and non-abraded samples followed by ultrasound cleaning. Fluorescent microscope images show that coating of HSA was successful even at lower concentrations of HSA and after several solvent cleaning.

In vitro confocal images of colonization of Staphylococcus aureus onto an HSA coated titanium substrate through wet coating procedures shown in FIG. 14 (FIG. 29A) and a control Ti surface (FIG. 29B) shows significant amounts of live bacteria on the uncoated titanium surface while the albumin surface shows almost no bacterial adhesion. These confocal images indicate that the albumin based coating reduces the attachment of bacteria to the surface.

### Example 6

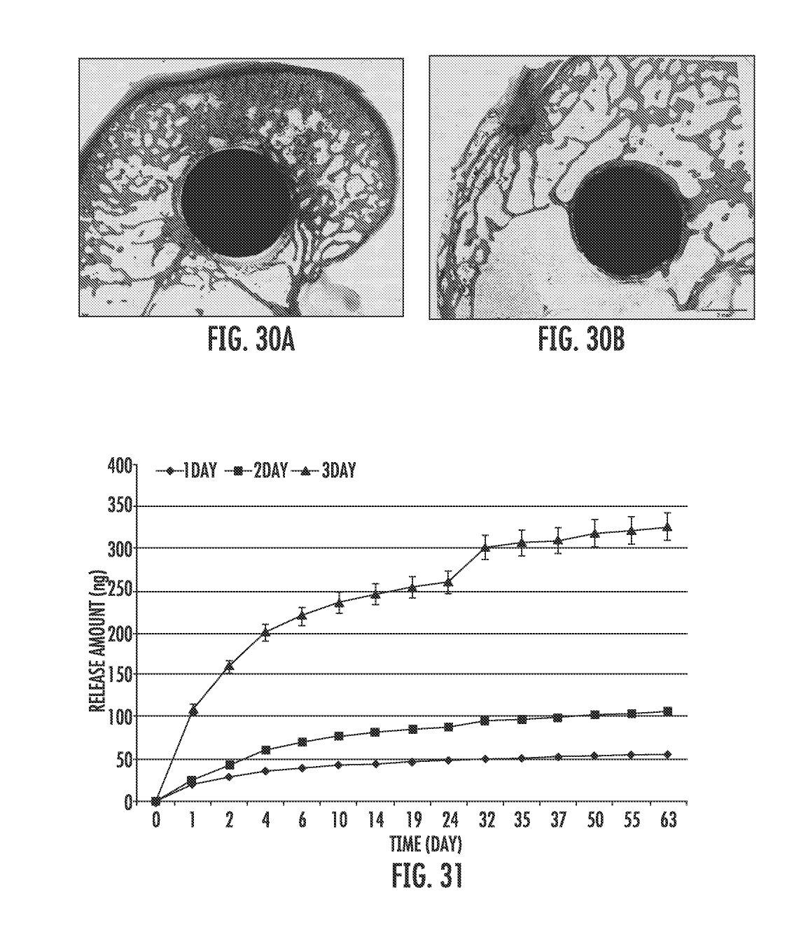

Bone response to human serum albumin plastic coated metal implant surfaces were evaluated in a rabbit distal-femoral-defect model. Three types of implants with the same morphology were examined: Group I titanium implants coated with a consolidated protein-based coating material; Group II titanium implants coated with an albumin solution; and Group III uncoated titanium implants. Sterilized implants were placed in the distal lateral femoral condyles of New Zealand White adult rabbits and tested at three time points (4, 12, and 20 weeks). Distal femurs containing implants were sectioned and imaged. Sections were stained with Cole's hematoxylin and eosin, Goldner-Masson trichrome stain, and the von Kossa stain. FIG. 30A illustrates a sample from Group I and

FIG. 30B illustrates a sample from Group III. Both albumin plastic coated and uncoated titanium implants form osteo-integration with surrounding bone tissue, which indicates that albumin plastic coating does not compromise the implant-bone tissue integration.

### Example 7

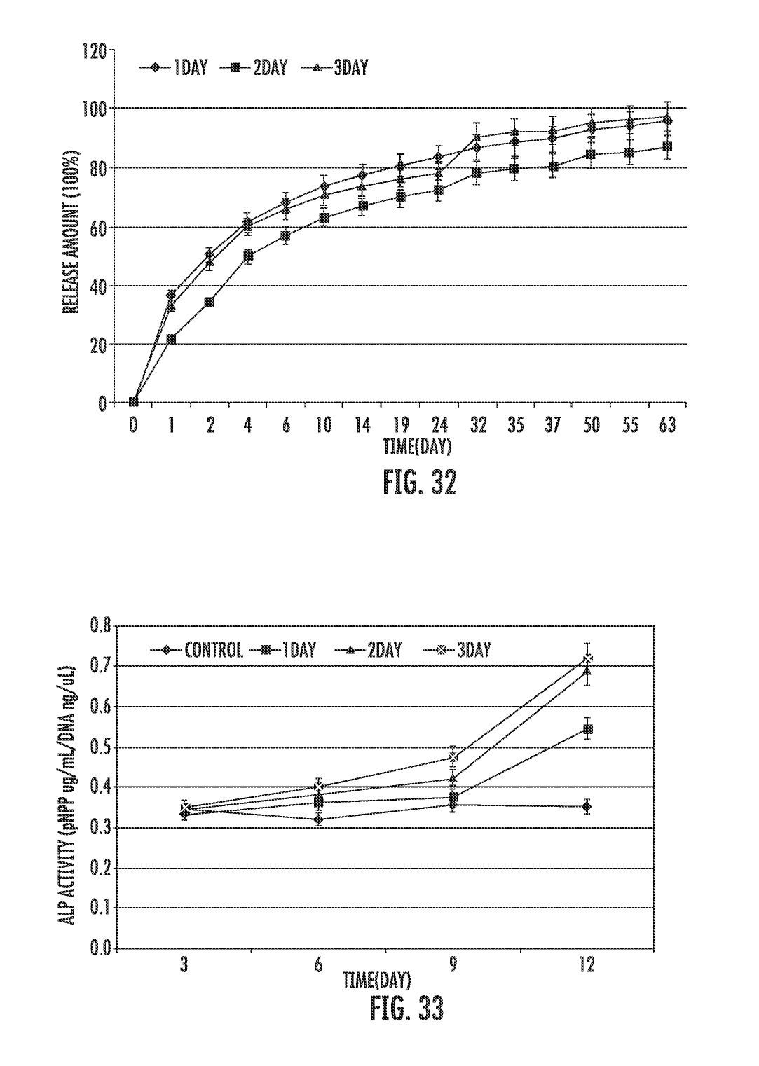

Albumin plastic coated implants were immersed in BMP2 containing solution for different time, ranging from 1 day to 3 days. After drying for 24 hours under vacuum, the implants were cultured in PBS to examine BMP2 release using ELISA. As shown in FIGS. 31 and 32, BMP2 release may last for several weeks. The BMP2 release was significantly longer from the implants immersed in BMP2 solution for 3 days than the implants immersed for 1 or 2 days. To further evaluate the bioactivity of the released biomolecules from albumin plastic, such as BMP2, the osteoblasts were cultured with BMP2 loaded albumin plastic coated implants for 12 days and ALP assay was used to evaluate the bioactivity of the release BMP2, the data in FIG. 33 shown that BMP2 release from albumin plastic coating is bioactive.

While the subject matter has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present disclosure should be assessed as that of the appended claims and any equivalents thereto.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed:

1. A medical device comprising:
a plastic albumin body having a defined three dimensional shape.

2. The medical device of claim 1, wherein the plastic albumin body is a unitary substantially monolithic body.

3. The medical device of claim 1, wherein the plastic albumin body has an antibacterial property such that it is resistant to bacterial adhesions and/or bacterial biofilm formation thereon.

4. The medical device of claim 1, wherein the plastic albumin body comprises at least one therapeutic agent.

5. The medical device of claim 1, wherein the plastic albumin body is sized and configured as a pressure equalization tube for an ear.

6. The medical device of claim 1, wherein the plastic albumin body is substantially rigid and has a substantially medial extending through channel.

7. The medical device of claim 1, wherein the plastic albumin body comprises cooperating discrete components.

8. The medical device of claim 1, wherein the plastic albumin body is an overmold or coating on a substrate.

9. The medical device of claim 1, wherein the medical device is a biodegradeable pressure equalization (PE) tube for an ear.

10. The medical device of claim 9, wherein the plastic albumin resides on a pre-molded PE tube substrate formed of a different material.

11. The medical device of claim 10, wherein the PE tube substrate comprises TEFLON.

12. The medical device of claim 1, wherein the medical device is an implantable device.

13. A method of fabricating a device, comprising;
introducing a liquid solution of albumin into a mold having a mold cavity with a defined shape;
heating the liquid solution in the mold for a defined time and temperature; and
forming a device having a solid plastic albumin shape in response to the heating step.

14. The method of claim 13, wherein the introducing step is carried out using a liquid solution having a concentration of albumin between about 5% to about 90%.

15. The method of claim 13, wherein the heating step is carried out by placing the mold in an autoclave at between about 80 degrees Celsius and 200 degrees Celsius for between about 10 minutes to 4 hours.

16. The method of claim 13, wherein the introducing step is carried out by providing powder albumin that is dissolved in a liquid and mixed with at least one therapeutic agent.

17. The method of claim 13, wherein the device is a medical device, the method further comprising applying at least one therapeutic agent onto the albumin before, during or after the forming step.

18. The method of claim 13, further comprising placing a rod in the mold before the introducing step to be in communication with a respective mold cavity, wherein the introducing step introduces the liquid under pressure into the mold about the rod, and wherein the forming step is carried out to forms a through channel in the solid albumin body with a diameter corresponding to a diameter of the rod.

19. The method of claim 13, further comprising placing a substrate in the mold before the introducing step to be in communication with a respective mold cavity, wherein the introducing step introduces the liquid under pressure into the mold about the substrate, and wherein the forming step is carried out to overmold the plastic albumin onto a surface or surfaces of the substrate.

20. A method of treating a patient, comprising;
providing a tube with an open channel, the tube comprising a substrate with a plastic albumin coating or overmold or a tube having a plastic albumin substrate defining the tube;
placing the tube in a target location of a patient; and
providing at least one of a (i) drainage channel (ii) access path or (ii) vent or air path; or (iii) tissue support using the tube.

21. The method of claim 20, wherein the placing step comprises placing the tube as a pressure equalization tube in an ear of the patient.

22. A method of treating substrates to provide anti-bacterial properties, comprising:
providing a target substrate;
applying liquid albumin to the target substrate; and
heating the substrate with the applied liquid albumin to transform the albumin into plastic albumin on the target substrate.

23. The method of claim 22, wherein the target substrate is a pressure equalization (PE) tube.

24. The method of claim 22, wherein the target substrate is a device used in a patient or baby care facility.
